(19) United States
(12) Patent Application Publication
(10) Pub. No.: US 2021/0000895 A1
(43) Pub. Date: Jan. 7, 2021

(54) EXOSOME TARGETING OF CD4+ EXPRESSING CELLS

(71) Applicant: Rhode Island Hospital, Providence, RI (US)

(72) Inventors: Bharat Ramratnam, Tiverton, RI (US); Xiaoli Tang, Barrington, RI (US)

(21) Appl. No.: 16/976,277

(22) PCT Filed: Mar. 1, 2019
(86) PCT No.: PCT/US19/20196

§ 371 Date: Aug. 27, 2020

Related U.S. Application Data
(60) Provisional application No. 62/637,336, filed on Mar. 1, 2018.

(51) Int. Cl. A61K 35/76; C07K 14/54; C07K 14/705; C07K 14/16

(52) U.S. Cl. CPC A61K 35/76; C07K 14/5446; C07K 2319/10; C07K 14/163; C07K 2319/09; C07K 14/70596

(57) ABSTRACT

Provided herein are compositions, methods, and kits for delivering therapeutic agents to specific cell types. For example, antiviral agents or other drugs are targeted to CD4+ T cells.

### RELATED APPLICATIONS

This application claims the benefit of priority under 35 U.S.C. § 119(e) to U.S. Provisional Application No. 62/637,336 filed Mar. 1, 2018, the entire contents of which is incorporated herein by reference.

### GOVERNMENT SUPPORT

This invention was made with government support under Grant No. K24HD80539, awarded by the National Institutes of Health. The government has certain rights in the invention.

### INCORPORATION-BY-REFERENCE OF SEQUENCE LISTING

The contents of the sequence listing text file named “21486_637001WO_ST25.txt”, which was created on Feb, 27, 2019 and is 53,248 bytes in size, is hereby incorporated by reference in its entirety.

### BACKGROUND

Early attempts used biologics such as OKT3 but were accompanied by adverse effects such as global immune stimulation and prolonged levels of CD4+T lymphocyte depletion. Some efforts have centered on the identification of Latency Reversal Agents (LRA) that activate virus without immune stimulation.

### SUMMARY OF THE INVENTION

The invention provides a solution to the long standing problems and drawbacks associated with targetting therapeutic agents to specific cell types, e.g. antiviral agents or other drugs to CD4+T cells. For example, the invention features a composition comprising an exosome that comprises a surface-exposed interleukin-16 (IL-16) polypeptide, e.g., a lysosomal-associated membrane protein (lamp)/IL-16 fusion protein. In preferred embodiments, the IL-16 polypeptide comprises the amino acid sequence of RRKS. An exemplary lamp protein comprises Lamp2b. The exosome optionally further comprises a latency reversal agent (LRA) such as an HIV Tat polypeptide. Optionally, the exosome comprises a nuclear localization signal such as a myc sequence.

The exosome is characterized as having a diameter from about 10 nm to about 5000 nm, from about 10 nm to about 1000 nm, e.g., a diameter from about 10 nm to about 300 nm, from about, from about 30 nm to about 150 nm, or from about 30 nm to about 100 nm.

Also within the invention is a method for promoting viral transcription in a cell by contacting an HIV-infected CD4+T cell with the composition described above.

Methods for preparing an exosome comprising a surface-exposed interleukin-16 (IL-16) polypeptide are also encompassed. For example, the method includes the steps of culturing cells, e.g., eukaryotic cells, in a medium, wherein the cells release the exosomes by secretion into the medium, collecting the supernatant of medium, fractionating the supernatant comprising the exosomes, and isolating the exosomes. For example, the fractionating can include separation methods comprising centrifugation (e.g., density centrifugation) or immunological methods (e.g., antibody beads). Additional methods include ultracentrifugation, ultrafiltration, polymer-based reagents, size exclusion chromatography, density gradient separation, and immunoaffinity capture.

The compositions and methods are useful for treating subjects, e.g., human patients, that have been diagnosed as being infected with human immunodeficiency virus-1 (HIV-1). Such therapeutic methods include the steps of administering to the individual an effective amount of a pharmaceutical composition comprising an exosome, wherein the exosome comprises a surface-exposed interleukin-16 (IL-16) polypeptide. For clinical use, adeno-associated virus (AAV) encoding the engineered LRA (Exo-Tat) will be injected intravenously into HIV infected patients. Exosomes will generate by AAV infected cells in the body. The AAV may be administered in a range from about 1×10⁹ to about 2×10⁹ genomic copies/mouse. Alternatively, the AAV may be administered in an amount equivalent to a protein standard.

Kits that include one or more reagents for preparing an exosome, wherein the exosome comprises a surface-exposed interleukin-16 (IL-16) polypeptide and a latency reversal agent are also within the invention.

Thus, provided and described herein are compositions, methods, and kits for antiviral therapies. In embodiments, provided herein is a composition comprising an exosome. In embodiments, the exosome comprises a surface-exposed interleukin-16 (IL-16) polypeptide.

In embodiments, the exosome comprises a lysosomal-associated membrane protein (lamp)/IL-16 fusion protein. In embodiments, the lamp protein comprises Lamp2b.

In embodiments, the exosome further comprises a latency reversal agent (LRA). In embodiments, the latency reversal agent comprises an HIV Tat polypeptide.

In embodiments, the IL-16 polypeptide comprises the amino acid sequence of RRKS (SEQ ID NO: 1).

In embodiments, the exosome comprises a nuclear localization signal (NLS). In examples, the NLS comprises c-myc (PAAKRVKLD SEQ ID NO: 2), nucleoplasmin (AVKRPAATKKAGQAKKKKLD SEQ ID NO: 3), EGL-13 (MSRRRKANPTKLSENAKKLAKEVEN SEQ ID NO: 4), TUS-protein (KLKIKRPVK SEQ ID NO: 5), or HIV-1 Tat (GRKKRRQRRRAP SEQ ID NO: 6), or HIV-1 Tat (RKKRRQRRR) (SEQ ID NO: 28). In embodiments, the nuclear localization signal comprises myc. The sequence of c-Myc nuclear localization signal comprises PAAKRVKLD (SEQ ID NO: 2).

In embodiments, the exosome has a diameter from about 10 nm to about 5000 nm, from about 10 to about 1000 nm. In embodiments, the exosome has a diameter from about 30 nm to about 100 nm.

In embodiments, provided herein are methods for promoting viral transcription in a cell, the method comprising contacting an HIV-infected CD4+T cell with the composition of described herein.

In embodiments, provided herein are methods for preparing an exosome comprising a surface-exposed interleukin-16 (IL-16) polypeptide. In embodiments, the method comprises, culturing cells in a medium, wherein the cells release the exosomes by secretion into the medium, collecting the supernatant of medium, fractionating the supernatant comprising the exosomes, and isolating the exosomes. For example, the fractionating can include separation methods comprising centrifugation (e.g., density centrifugation) or immunological methods (e.g., antibody beads). Additional methods include ultracentrifugation, ultrafiltration, polymer-based reagents, size exclusion chromatography, density gradient separation, and immunoaffinity capture.

In embodiments, the cells comprise eukaryotic cells. In embodiments, the cells comprise a nucleic acid encoding for a protein of interest. In embodiments, the protein of interest comprises a viral protein (e.g., HIV Tat).

In embodiments, provided herein are methods of treating a patient comprising human immunodeficiency virus-1 (HIV). In embodiments, the method comprises administering to the patient an effective amount of a pharmaceutical composition comprising an exosome, wherein the exosome comprises a surface-exposed interleukin-16 (IL-16) polypeptide (T I V I R R K S L Q S K E T T A A G D S) (SEQ ID NO: 7). The IL-16 polypeptide comprises residues from the binding domain. In embodiments, the IL-16 polypeptide comprises at least 10 amino acids, at least 15 amino acids, or at least 20 amino acids. Advantageously, the IL-16 polypeptide is endogenous, and thus will not elicit an immune response.

In embodiments, patient is administered the composition intravenously.

In embodiments, the patient comprises a human. In embodiments, the effective amount is an amount effective to promote viral transcription. In embodiments, the effective amount is from about 0.01 ng to about 10,000 ng of the composition. In embodiments, the effective amount is from about 0.01 ng/mL to about 10,000 ng/mL of the composition.

Provided herein are kits comprising one or more reagents for preparing an exosome. In embodiments, the exosome comprises a surface-exposed interleukin-16 (IL-16) polypeptide and a latency reversal agent.

Other features and advantages of the invention will be apparent from the following description of the preferred embodiments thereof, and from the claims. All references, including GENBANK™ references or other sequence references, cited herein are incorporated by reference.

### BRIEF DESCRIPTION OF THE DRAWINGS

FIG. 1A is a photograph of a Western blot that revealed robust expression of Tat in cellular lysates but not in released exosomes. An empty expression vector (EV) was used as a control. Using an expression vector encoding wild-type HIV-1 Tat (pTat), Protein levels in cell lysates of transfected cells were profield as well as in released exosomes of 30-150 nm in diameter. The exosome marker Alix was used to control for protein loading. FIG. 1B is a photograph of a Western blot of transfected cells which revealed Tat protein expression in both cellular lysates and exosomal preparations. The Tat expression vector was modified to include a peptide sequence that targets proteins to the interior exosomal membrane (pXO-Tat). No Tat protein was detected with use of the empty expression vector (EV). The exosome marker Alix was used to control for protein loading. FIG. 1C is a bar graph showing that exosomal localization (pXO-Tat) decreased transactivating activity, which activity depended upon nuclear localization of Tat. The TZM-bl cell line (NIH AIDS Reagent Program Cat.#8129) was used to quantify the transactivating activity of Tat produced by wild type (pTat), exosomal localization modified-(pXO-Tat) and nuclear/exosomal localization modified-(pEXO-Tat) expression vectors. FIG. 1D is a bar graph that depicts that the inclusion of a cmyc nuclear localization signal (NLS) increased activity to ˜50% of wild-type Tat levels (pTat). FIGS. 1A-1D depict exosomal localization and biological activity of modified HIV-1 Tat expression vectors.

FIG. 2A is a photograph of a Western blot of EXO-Tat protein expression in whole cell lysate. FIG. 2B is a photograph of a Western blot of EXO-Tat protein expression in the cytoplasmic fraction. FIG. 2C is a photograph of a Western blot of EXO-Tat protein expression in the nuclear fraction. FIG. 2D is a photograph of a Western blot of EXO-Tat protein expression in the membranous fraction. FIG. 2E is a photograph of a Western blot of EXO-Tat protein expression in the released exosome. FIGS. 2A-2E show the subcellular localization of engineered EXO-Tat protein. HEK293 cells were maintained in culture and transfected by pEXO-Tat or an empty expression vector (EV). Cells and supernatants were fractionated and Western blot revealed Tat expression in all cellular fractions examined as well as in released exosomes. GAPDH was used to control for protein loading in experiments involving cellular fractions and Alix was used for those involving exosomal preparations. No Tat protein was detected in parallel experiments involving the transfection of an empty expression vector (EV).

FIG. 3A is a bar graph that depicts results from the transfection of pEXO-Tat into U1 cells that harbored integrated HIV-1, which led to increased virion production and release as quantified by HIV-1 p24 antigen levels in culture supernatant over a 48-hour period. P24 levels were normalized to values obtained with control experiments involving the transfection of an empty expression vector (EV). FIG. 3B is bar graph that depicts the result of the J-Lat GFP (clone A72) cell line which allowed quantification of viral promoter (LTR) activation by GFP expression. Transfection of pEXO-Tat into J-Lat GFP cells led to 3-fold increase in GFP expression compared to experiments involving the empty expression vector (EV). Blot band intensity was measured using the Licor Odyssey software. FIGS. 3A and 3B are bar graphs showing that EXO-Tat activated the HIV-1 LTR promoter in vitro models of viral latency. Two well characterized cellular models were used to quantify the effect of EXO-Tat on viral activity.

FIG. 4A is a photograph of a Western blot depicting a stable cell line expressing EXO-Tat that was generated by transducing HEK293 T cells with EXO-Tat lentiviruses and screened under the pressure of puromycin (Puro). After being cultured in 5 μg/ml Puro for 15 days, the cells expressed stable levels of Tat protein. FIG. 4B is an image depicting the quantification of CD4+T lymphocyte uptake, where exosomes were isolated and labeled with the lipophilic dye DIO. Incubation of labeled exosomes with CD4+T lymphocytes led to 13% of cells acquiring dye, as quantified by flow cytometry. Unlabeled exosomes served as a control. FIG. 4C is a photomicrographic image that depicts the visualized exosome association with CD4+T lymphocytes, fluorescent dye conjugated antibody was used which recognizes EXO-Tat protein. A representative CD4+T lymphocyte with EXO-Tat exosomes is shown. FIGS. 4A-4C show the generation of exosomes loaded with Tat protein and their association with CD4+T cells.

FIG. 5A is a bar graph showing that EXO-Tat exosomes reactivated latent HIV-1 and increased its mRNA expression in cells. FIG. 5B is a bar graph depicting that EXO-Tat exosomes increased the release of HIV-1 mRNA into culture medium as detected by RT-qPCR. FIG. 5C is a dot plot graph depicting that EXO-Tat reactivated replication competent HIV-1 as measured by p24 concentration in the supernatants of 3/6 patient samples. FIG. 5D is a graph depicting that EXO^{CD4}-Tat exosomes reactivated replication competent HIV-1 from 3/3 patient samples. The lower limit of p24 quantification is 0.0075 μg/mL. FIGS. 5A-5D depict data that EXO-Tat exosomes reactivated latent HIV-1 ex vivo in resting (r)CD4+T cells. rCD4+T cells isolated from the PBMCs of ART treated patient blood; two million rCD4+T cells were treated with control exosomes (Exo-C), EXO-Tat exosomes (EXO-Tat) or PMA/I respectively for 4 days. The cells and supernatants were separated by centrifugation. HIV-1 mRNA was determined by real-time RT-qPCR. P24 concentration in the supernatants was measured by ELISA.

FIG. 6A is a photograph of a Western blot that depicts that the inclusion of a CD4+ binding moiety in exosomes (EXO^{CD4}-Tat) increased CD4+T cell binding. Control Exosomes (Exo-C), EXO-Tat or EXO^{CD4}-Tat exosomes were incubated with CD4+T cells for 24 h and Western blot was used to compare intracellular Tat levels in cells treated with EXO-Tat or EXO^{CD4}-Tat exosomes. GAPDH was used as a cell lysate loading control. WB band intensity was measured using the Licor Odyssey software. FIG. 6B is a photomicrographic image depicting that EXO^{CD4}-Tat exosomes specifically targeted CD4+T cells. Exo-C, EXO-Tat or EXO^{CD4}-Tat exosomes that were incubated with PMBCs from healthy donor for 24 h. The supernatants were removed by centrifugation. The cell pellets were prepared and probed with fluorescent conjugated antibodies which recognize CD4 (green) or HA-tagged Tat (red). The top panel shows CD4 staining (green). The middle panel shows CD4 staining (green), Tat staining (red) and the merge of green and red. The bottom panel shows CD4 staining (green), Tat staining (red) and the merge of green and red. The much stronger merged color orange indicates Tat protein containing exosomes binding to CD4+T cells. FIGS. 6A and 6B depict that EXO′-Tat exosomes specifically targeted CD4+cells.

FIG. 7A is a bar graph depicting the expression levels of T cell activation markers CD25, CD69 and HLA-DR as measured by flow cytometry. EXO^{CD4}-Tat exosomes had no impact on those markers. FIG. 7B is a bar graph depicting the expression levels of pro-inflammatory cytokines (IL-1α, IL-1(3, IL-2, IL-4, IL-6, IL-8, IL-10, IL-12, IL-17α, IFN-γ, TNF-α, and GM-CSF) in the supernatants as measured using a Multi-Analyte ELISArray Kit. EXO^{CD4}-Tat exosomes had no significant effect on the release of pro-inflammatory cytokines. PMA/I significantly increased the release of IL-2, IL-4, IL-6, IL-8, IL-10, IL-17α, IFN-γ, TNF-α and GM-CSF. All experiments were performed in duplicate. FIGS. 7A and 7B are bar graphs that depict the effect of EXO^{CD4}-Tat exosomes on T cell activation and cytokine release. Resting CD4+T cells were isolated from PBMCs of healthy donors and treated with control exosomes (Exo-C), EXO^{CD4}-Tat exosomes or PMA/I for 2 days. The cells and supernatants were separated by centrifugation.

FIG. 8 is a graph depicting that combining Exo-Tat with CD4-αCD3HA eliminated HIV-1+cells. Five million T cells from the blood of cART-treated HIV-1 infected individuals were cultured in control or CD4-αCD3HA medium supplemented with penicillin-streptomycin, L-glutamine, 0.1 nM IL-7, 1 μM tenofovir, 1 μM nevirapine, 1 μM emtricitabine, 10 μM T20, and 10 U/ml DNAse I (ProSpec) and treated with Exo-C exosomes (4 days), Exo-Tat exosomes (4 days) or PMA/I (18 hr). On day 5, 2.5 million T cells were cocultured with 2 million MOLT-4 cells in RPMI medium supplemented with 2 μg/ml phytohemagglutinin and radiated PBMCs for 14 days. The culture supernatants were used for measuring p24 level using the simoa technology at Quanterix. N=5, p<0.001.

### DETAILED DESCRIPTION

Highly active antiretroviral treatment (HAART) of HIV-1 eliminates productively infected cells with plasma levels of virus being reduced to levels below the limit of detection of current assays. While treatment leads to the reduction in total body viral burden, a relatively small but stable reservoir of latently infected cells remains. When HAART is stopped, stochastic activation of infected reservoir cells eventually leads to plasma viremia in many individuals. Thus, a major roadblock to HIV-1 cure is the inability to eliminate latently infected cells despite prolonged antiretroviral therapy.

### Therapy to Target Residual Reservoir of HIV-1 Infected CD4+Lymphocytes

Transcriptional activation of latent HIV-1 has served as a potential strategy to purge viral reservoirs with the hope that productively infected cells will succumb to viral cytopathicity or immune clearance under the cover of antiretroviral therapy. However, replication competent HIV-1 persists in a subpopulation of CD4+T lymphocytes despite prolonged antiretroviral treatment. This residual reservoir of infected cells harbors transcriptionally silent provirus capable of reigniting productive infection upon discontinuation of antiretroviral therapy. Certain classes of drugs can activate latent virus but not at levels that lead to reductions in HIV-1 reservoir size in vivo. The utility of CD4+ receptor targeting exosomes as an HIV-1 latency reversal agent (LRA) is provided. Human cellular exosomes were engineered to express HIV-1 Tat, a protein that is a potent transactivator of viral transcription. Preparations of exosomal Tat activated HIV-1 in primary, resting CD4+T lymphocytes were isolated from antiretroviral treated individuals with prolonged periods of viral suppression and led to the production of replication competent HIV-1.

Exosomal Tat is useful as a biologic product with utility in targeting latent HIV-1 and treating HIV-1 infected patients, thereby conferring a clinical benefit.

### Exosome and a Surface-Exposed Interleukin-16 (IL-16) Polypeptide.

Exosomes were first described as a means for reticulocytes to selectively discard transferrin receptors as they matured into erythrocytes. For a long time thereafter, they were seen as a means for the removal of unwanted cellular components. B cells shed exosomes containing antigen-specific MHC II capable of inducing T cell responses and these small vesicles may be involved in a multitude of functions, both physiological and pathological. Exosomes are small membrane-bound vesicles of endocytic origin that are released into the extracellular environment following fusion of multivesicular bodies with the plasma membrane. Exosomes generally range in size from between about 10 nm to about 5000 nm, and 10 nm to about 1000 nm in diameter. For example, exosomes have a diameter between about 10 nm and 900 nm, between about 10 nm and about 800 nm, between about 10 nm and about 700 nm, between about 10 nm and about 600 nm, between about 10 nm and about 500 nm, between about 10 nm and about 400 nm, between about 10 nm and about 300 nm, between about 10 nm and about 200 nm, between about 10 nm and about 100 nm, between about 10 nm and about 50 nm. In preferred embodiments, the exosomes have a diameter from about 10 nm to about 300 nm, from about 30 nm to about 150 nm, or from about 30 nm to about 100 nm.

The invention provides exosomes loaded with one or more exogenous protein and/or peptide. Exosomes are prepared and then loaded with the desired protein and/or peptide for delivery (e.g., IL-16 and/or an IL-16/Lamp2b fusion protein). The protein or peptide can be loaded in the exosomes by expression or overexpression of the protein or peptide in the cell which is used to produce the exosomes.

The term “exogenous” refers to a protein with which the cell or exosome is not normally associated or expresses in its native or wild type state.

An exemplary exogenous protein and/or peptide is an IL-16 protein. For example, the IL-16 is human IL-16 and comprises the amino acid sequence, or fragment thereof (the polypeptide may comprise the underlined residues):

| | |
|---|---|
| 1 | mdysfdttae dpwvrisdci knlfspimse nhghmplqpn aslneeegtq ghpdgtppkl |
| 61 | dtangtpkvy ksadsstvkk gppvapkpaw frqs1kglin rasdprglpd palstqpapa |
| 121 | srehlgshir assssssirq rissfetfgs sqlpdkgaqr lslqpssgea akplgkheeg |
| 181 | rfsgllgrga aptivpqqpe qvlssgspaa seardpgvse sppprrqpnq ktlppgpdpl |
| 241 | lrllstqaee sqgpvlkmps qrarsfpltr sqscetklld ektsklysis sqvssavmks |
| 301 | llclpssisc aqtpcipkeg asptsssned saangsaets aldtgfslnl selreytegl |
| 361 | teakedddgd hsslqsgqsv isllsseelk klieevkvld eatlkqldgi hvtilhkeeg |
| 421 | aglgfslagg adlenkvitv hrvfpnglas qegtiqkgne vlsingkslk gtthhdalai |
| 481 | lrdareprqa vivtrkltpe ampdlnsstd saasasaasd vsvestaeat vctvtlekms |
| 541 | aglgfslegg kgslhgdkpl tinrifkgaa seqsetvqpg deilqlggta mqgltrfeaw |
| 601 | niikalpdgp vtivirrksl qskettaagd s |

SEQ ID NO: 8; GenBank Accession AAB58261.1, incorporated herein by reference.

Exemplary landmark residues, domains, and fragments of IL-16 include, but are not limited to residues 347-432 (crotonase like domain), residues 410-487 (PDZ signaling domain), residues 533-619 (PDZ signaling domain) A fragment of an IL-16 protein is less than the length of the full length protein, e.g., a fragment is at least 3, 4, 5, 6, 7, 8, 9, 10, 20, 30, 40, 50, 100, 200 or more residues in length, but less than e.g., 631 residues in the case of IL-16 above. For example, an IL-16 fragment comprises or consists of 2-20 residues, e.g., 2-10 residues e.g. 3-5 residues, e.g. 4 residues. Alternatively, an IL-16 fragment comprises of at least 10 residues, at least 15 residues, at least 20 residues, or at least 30 residues. For example, the IL-16 polypeptide comprises the amino acid sequence T I V I R R K S L Q S K E T T A A GDS (SEQ ID NO: 7), underlined above.

In embodiments, the IL-16 polypeptide comprises the amino acid sequence of RRKS (SEQ ID NO: 1).

In embodiments, the human IL-16 nucleotide sequence is depicted below. The start and stop codons are bold and underlined.

| | |
|---|---|
| 1 | ctgctgctac cacaggaaga cacagcaggg agaagcccta gtgcctctgc cggctgccca |
| 61 | ggacctggta tcggcccaca gaccaagtcc tccacagagg gcgagccagg gtggagaaga |
| 121 | gccagcccag tgacccaaac atccccgata aaacacccac tgcttaagag gcaggctcgg |
| 181 | atggactata gatttgatac cacagccgaa gacccttggg ttaggatttc tgactgcatc |
| 241 | aaaaacttat ttagccccat catgagtgag aaccatggcc acatgcctct acagcccaat |
| 301 | gccagcctga atgaagaaga agggacacag ggccacccag atgggacccc accaaagctg |
| 361 | gacaccgcca atggcactcc caaagtttac aagtcagcag acagcagcac tgtgaagaaa |
| 421 | ggtcctcctg tggctcccaa gccagcctgg tttcgccaaa gcttgaaagg tttgaggaat |
| 481 | cgtgcttcag acccaagagg gctccctgat cctgccttgt ccacccagcc agcacctgct |
| 541 | tccagggagc acctaggatc acacatccgg gcctcctcct cctcctcctc catcaggcag |
| 601 | agaatcagct cctttgaaac ctttggctcc tctcaactgc ctgacaaagg agcccagaga |
| 661 | ctgagcctcc agccctcctc cggggaggca gcaaaacctc ttgggaagca tgaggaagga |
| 721 | cggttttctg gactcttggg gcgaggggct gcacccactc ttgtgcccca gcagcctgag |
| 781 | caagtactgt cctcggggtc ccctgcagcc tccgaggcca gagacccagg cgtgtctgag |
| 841 | tcccctcccc caaggcggca gcccaatcag aaaactctcc cccctggccc ggacccgctc |
| 901 | ctaaggctgc tgtcaacaca ggctgaggaa tctcaaggcc cagtgctcaa gatgcctagc |
| 961 | cagcgagcac ggagcttccc cctgaccagg tcccagtcct gtgagacgaa gctacttgac |
| 1021 | gaaaagacca gcaaactcta ttctatcagc agccaagtgt catcggctgt catgaaatcc |
| 1081 | ttgctgtgcc ttccatcttc tatctcctgt gcccagactc cctgcatccc caaggaaggg |
| 1141 | gcatctccaa catcatcatc caacgaagac tcagctgcaa atggttctgc tgaaacatct |
| 1201 | gccttggaca cagggttctc gctcaacctt tcagagctga gagaatatac agagggtctc |
| 1261 | acggaagcca aggaagacga tgatggggac cacagttccc ttcagtctgg tcagtccgtt |
| 1321 | atctccctgc tgagctcaga agaattaaaa aaactcatcg aggaggtgaa ggttctggat |
| 1381 | gaagcaacat taaagcaatt agacggcatc catgtcacca tcttacacaa ggaggaaggt |
| 1441 | gctggtcttg ggttcagctt ggcaggagga gcagatctag aaaacaaggt gattacggtt |
| 1501 | cacagagtgt ttccaaatgg gctggcctcc caggaaggga ctattcagaa gggcaatgag |
| 1561 | gttctttcca tcaacggcaa gtctctcaag gggaccacgc accatgatgc cttggcaatc |
| 1621 | ctccgccaag ctcgagagcc caggcaagct gtgattgtca caaggaagct gactccagag |
| 1681 | gccatgcctg acctcaactc ctccactgac tctgcagcct cagcctctgc agccagtgat |
| 1741 | gtttctgtag aatctacagc agaggccaca gtctgcacgg tgacactgga gaagatgtcg |
| 1801 | gcagggctgg gcttcagcct ggaaggaggg aagggctccc tacacggaga caagcctctc |
| 1861 | accattaaca ggattttcaa aggagcagcc tcagaacaaa gtgagacagt ccagcctgga |
| 1921 | gatgaaatct tgcagctggg tggcactgcc atgcagggcc tcacacggtt tgaagcctgg |
| 1981 | aacatcatca aggcactgcc tgatggacct gtcacgattg tcatcaggag aaaaagcctc |
| 2041 | cagtccaagg aaaccacagc tgctggagac tcctag |

SEQ ID NO: 9 GenBank accession number: U82972.1, incorporated herein by reference. For example, the nucleic acid sequence encoding the IL-16 polypeptide comprises: acgattgtca tcaggagaaa aagcctccag tccaaggaaa ccacagctgc tggagactcc SEQ ID NO: 10.

An exemplary engineered exosome comprises a lysosomal-associated membrane protein (Lamp)/IL-16 fusion protein. For example, the Lamp protein comprises Lamp2b.

Human Lamp2b protein and comprises the amino acid sequence, or fragment thereof:

| | |
|---|---|
| 1 | mvcfrlfpvp gsglvlvclv lgavrsyale lnitdsenat clyakwqmnf tvryettnkt |
| 61 | yktvtisdhg tvtyngsicg ddqngpkiav qfgpgfswia nftkaastys idsysfsynt |
| 121 | gdnttfpdae dkgiltvdel lairipindl frcnslstle kndvvqhywd vlvqafvqng |
| 181 | tvstneflcd kdktstvapt ihttvpsptt tptpkekpea gtysvnngnd tcllatmglq |
| 241 | lnitqdkvas vininpntth stgscrshta llrinsstik yldfvfavkn enrfylkevn |
| 301 | ismylvngsv fsiannnlsy wdaplgssym cnkeqtvsys gafqintfdl rvqpfnvtqg |
| 361 | kystaqdcsa dddnflvpia vgaalagvli lvllayfigl khhhagyeqf |

SEQ ID NO: 11; GenBank Accession P13473.2, incorporated herein by reference.

Exemplary landmark residues, domains, and fragments of Lamp2b include, but are not limited to residues 1-28 (signal peptide), residues 29-410 (mature protein), residues 29-375 (topological domain), residues 29-192 (lumenal domain), residues 38, 49, 58, 75, 101, 123, 179, 196, 200, 203, 207, 209, 210, 211, 213, 229, 257, 275, 300, 317, 356 (glycosylation sites), residues 374-377 (beta strand region), residues 376-399 (transmembrane region). A fragment of a Lamp2b is protein is less than the length of the full length protein, e.g., a fragment is 10, 20, 30, 40, 50, 100, 200 or more residues in length, but less than e.g., 410 residues in the case of Lamp2b above.

In embodiments, the Lamp2b nucleic acid sequence comprises the sequence below. The start and stop codon are bold and underlined.

| | |
|---|---|
| 1 | aagaaagagc cccgccccta gtcttatgac tcgcactgaa gcgccgattc ctggcttttg |
| 61 | caaggctgtg gtcggtggtc atcagtgctc ttgacccagg tccagcgagc cttttccctg |
| 121 | gtgttgcagc tgttgttgta ccgccgccgt cgccgccgtc gccgcctgct ctgcggggtc |
| 181 | atggtgtgct tccgcctctt cccggttccg ggctcagggc tcgttctggt ctgcctagtc |
| 241 | ctgggagctg tgcggtctta tgcattggaa cttaatttga cagattcaga aaatgccact |
| 301 | tgcctttatg caaaatggca gatgaatttc acagtacgct atgaaactac aaataaaact |
| 361 | tataaaactg taaccatttc agaccatggc actgtgacat ataatggaag catttgtggg |
| 421 | gatgatcaga atggtcccaa aatagcagtg cagttcggac ctggcttttc ctggattgcg |
| 481 | aattttacca aggcagcatc tacttattca attgacagcg tctcattttc ctacaacact |
| 541 | ggtgataaca caacatttcc tgatgctgaa gataaaggaa ttcttactgt tgatgaactt |
| 601 | ttggccatca gaattccatt gaatgacctt tttagatgca atagtttatc aactttggaa |
| 661 | aagaatgatg ttgtccaaca ctactgggat gttcttgtac aagcttttgt ccaaaatggc |
| 721 | acagtgagca caaatgagtt cctgtgtgat aaagacaaaa cttcaacagt ggcacccacc |
| 781 | atacacacca ctgtgccatc tcctactaca acacctactc caaaggaaaa accagaagct |
| 841 | ggaacctatt cagttaataa tggcaatgat acttgtctgc tggctaccat ggggctgcag |
| 901 | ctgaacatca ctcaggataa ggttgcttca gttattaaca tcaaccccaa tacaactcac |
| 961 | tccacaggca gctgccgttc tcacactgct ctacttagac tcaatagcag caccattaag |
| 1021 | tatctagact ttgtctttgc tgtgaaaaat gaaaaccgat tttatctgaa ggaagtgaac |
| 1081 | atcagcatgt atttggttaa tggctccgtt ttcagcattg caaataacaa tctcagctac |
| 1141 | tgggatgccc ccctgggaag ttcttatatg tgcaacaaag agcagactgt ttcagtgtct |
| 1201 | ggagcatttc agataaatac ctttgatcta agggttcagc ctttcaatgt gacacaagga |
| 1261 | aagtattcta cagctcaaga ctgcagtgca gatgacgaca acttccttgt gcccatagcg |
| 1321 | gtgggagctg ccttggcagg agtacttatt ctagtgttgc tggcttattt tattggtctc |
| 1381 | aagcaccatc atgctggata tgagcaattt tagaatctgc aacctgattg attatataaa |
| 1441 | aatacatgca aataacaaga ttttcttacc tctcagttgt tgaaacactt tgcttcttaa |
| 1501 | aattgatatg ttgaaacttt aattctttta tcaatcccag cattttgaga tcagtcttta |
| 1561 | ttaataaaac ctgttctctt taatcagctt aaaatccaaa gtgtcatatt tactggtcct |
| 1621 | ggagacaaac ttgttcaaaa gaacatcaac gtgcaatgtt ttaaggtcta tcttaagaag |
| 1681 | ccctggccaa attttgatcc taaccttgaa gtatgccttg aacttattaa catggccatt |
| 1741 | ataagaataa aatatgtagt tgtgtcttaa tggaattaat aaatgtcatt tcactactgg |
| 1801 | tgttctgttt caatgtataa ggactatagt gatttaaact catcaatgtg cctttgcata |
| 1861 | aagttcatta aataaatatt gatgtggtat aaatgcccat cagatatgct taaacttggt |
| 1921 | tttcagttga atgaagtaga gaatgtcctc aggaccatca gcattttaaa ggttatgtga |
| 1981 | cttttgctga tttctctgag ttcaagttaa gcatgaagtt agtacctcaa gcctgtgatt |
| 2041 | tttccctagg gatgatacag acccaagagg ctacaacaga acttaaactg gcttcgtaat |
| 2101 | tagagttttt aagataattg tttgtttttc agcaatatag actgaaaaga tccaagcata |
| 2161 | tttagccact tgcttttttg tttcttgttt tgttcttctt tggatgcctg attagtattg |
| 2221 | aaagatagaa atattctatg aactaattag gacagattgt gttgtgtttc tctacctcat |
| 2281 | cttgttgatc tctggagcat taaaatctat ttagtgttgt catcagtgtg gtacttatga |
| 2341 | aatgtaagct aacagcaatc tcagaaggga ggcagtgaag catagcaact aggctcttgt |
| 2401 | ttcttcaaga tggcccctgt ggggcagtgc atagatgggg gtgtaaagag aagctgttgg |
| 2461 | cattaaaatg agctagataa tcagcccttg ttgaagcata ttccatggta taagagtagc |
| 2521 | acagacatga aacatagata aagaaggaag gcttaataga ctagaagact tccacattga |
| 2581 | agtattatta acccattgta tgtatatagg ggcatgatca gagtctctat aacttcctga |
| 2641 | ttaacaatac agtgtatctt gttacccagc tgtcagtctt tgagagcttt cagtaaaata |
| 2701 | tagtaaattc tttcagcata ggctaatgtg tggttactga gatgagtgtt gtgtactcag |
| 2761 | aaccgtagca acatttttat gaatggtaaa agtacaagag gaggaaaagt taaaattaga |
| 2821 | agaaaagtac aagttattgc ttaatcataa atcacaccag ataacacatt ttgttaattt |
| 2881 | cattagctat tactggaaag gaccttaacg attatttaca gaaaggggag tgaaattcat |
| 2941 | tgaggttcca tatcaagtgg gcaacaaaac tattactagc attttgataa aaattgcccc |
| 3001 | taatgaaatc tagtcactca acagtaaaac aacagctggt ttacacttga aattatgaga |
| 3061 | tcagaattgg gcactttggg cttccgtact atgttttgct taagtttttt ttttaatact |
| 3121 | aatatgggct ttttcagtag taatatacca aaacacttct attttaatct ctgtttgcta |
| 3181 | cttcaaaacc taatcctcct cagatgggat catgagcata agaggaaaag agaagagaat |
| 3241 | gaatacttgt tgacctcttg atgtgtatca gatgctttag aaatgtaatt gtatttaatc |
| 3301 | ctcaaatacc ttataggtat tattatcccc ttcttacaga tgaggaaagt gaggcccagt |
| 3361 | ttaaataact tgcccaaggt cctttggcta gtactggaag gagtcaagat ttaaacacag |
| 3421 | ttctgtctga atccagaact caaaatctac attgcacatg ttgctttcct ggtggttcgg |
| 3481 | tggaatggac tgcaacgcat tagatactgc tgttattctt ccaggccacc gctcagctaa |
| 3541 | aaataattgt gtgtgtgtgt atatatatat atatgtacac acacacacat atatatacac |
| 3601 | acacacatat atatacacat atacatatat atacacatat atatacacac acatatatat |
| 3661 | gtatatatat actacattct tgatcctaag tcttttttaa cttaaatttt attacttata |
| 3721 | cagaattctt atttatactt taattatagg tgtgacgaag agaaagagag tagggaaata |
| 3781 | cacaggcagt ggttttaagt gtagatgatg gctccttaac ccagtgtcat tagataatca |
| 3841 | aacctaaagt cttcccatat taggcaagcg caattctcta ttttggaccc ttcccattct |
| 3901 | tcccttacct tctgcttttc gtactgagga atttcgtgtg attttagata aagtgataat |
| 3961 | gagatattga gcaaataaga aaatagaggt aatgctataa aaaactaagc tatgtacact |
| 4021 | ttcaaaatgc atgtttcttg catgcttttt actacttaat tgcattcttt gctaatttcc |
| 4081 | tttccttgct gtctgttctt ttctaacagc tgaagaatgt tctgctgact ctgacctcaa |
| 4141 | ctttcttatt cctgttgcag tgggtgtggc cttgggcttc cttataattg ttgtctttat |
| 4201 | ctcttatatg attggaagaa ggaaaagtcg tactggttat cagtctgtgt aatcagttaa |
| 4261 | atctagtgtt tgtttgtttt tttcaattag aagttacgtt tccattggct aaaagccagg |
| 4321 | acatgctgtg caatagattg tttaagatat gcagactaac ttcagtgagt tcctagctaa |
| 4381 | cttgggcatg agtacactta tttaagacaa aatatattag gaccaatttt tttctgtttt |
| 4441 | ttttcttcct ttgttaaagt ataattaaaa gaaaaattgt ggcttagaat tttttaagta |
| 4501 | aataatgatt ttaagcccct ggatccaatt atgaaagcat ttttgctgat gtgtaatttt |
| 4561 | atatgttaca gttacttata ttttactact ttgatgttat ttgcaaaatc aaaggtgtta |
| 4621 | aagaatttaa cttgcttcag gaaataaatt caagaacata gtggattcat tttcattggt |
| 4681 | ggcagacacg aaatttggtt catgataaga cttcctttcc ccacctcctg atcagcatta |
| 4741 | tttaaatctg tatttttctg ttagttaaga aagaaatggc ttcatgatat tgtatttaat |
| 4801 | agcaaaagtt tggctgtctt cttcattact gttaatagct actatatttt aacaaggaga |
| 4861 | tttctttttt tgttgttgtt gttctagagt ttggaatata ctgattatct cagacttgac |
| 4921 | atttatactg aaggatgaag taagacctcc agcttttttt aaaaaaggtg ttgatttgga |
| 4981 | acacctgtat gggttatggt ttattaaggt tatggtttag aaagtttttt tccctcagag |
| 5041 | ccttaacttg ttaagaaggt tcatttatcc tgcactgaaa acaaaaactc tatatacttt |
| 5101 | gtttgtgtgc ctcctgcact ctcccattcc ctatgtgaat atgctctagt tgatattttt |
| 5161 | aatatattga tttctttttt ctcacagcaa caagtgctta ctctagaggt tagtgggccc |
| 5221 | tgatatgtca tcagtcagat gcctgcctag ccaaagctgg actaagatta ttctgtacat |
| 5281 | ttgttgatct tgatatagac ttatatccct gtagggactg ctaatggctc cggcttctgg |
| 5341 | agtaaggtac tggagaccac tcatccctgt gtctgcttga ttggttcagc tgttgaattg |
| 5401 | cccttttatt tggaagcagt gttgaagttg tctagggttc aaatggctgc tttgtacacc |
| 5461 | tgtcattagt ataaggcaga tgtttatttt atcaagctat tttatctcta catttaacta |
| 5521 | aaaacaaaag ttcccaaaga tctgccttca cttcagaaat tttttttgga ttaaaaaaat |
| 5581 | taagcctgaa ccttaaataa agtgagttgg ttattcattc caaggattaa gtcccaatct |
| 5641 | acctctcagc acaatgcaga agctcaccac tgtattgctg ccattaactc atgccagaac |
| 5701 | cctttgccaa taactggaat tacaaatttt tgttaaagaa aatttatcaa gatctttctt |
| 5761 | tactgccttc tctatatgta catctcaaaa acatgtacat ctcaaaaact ggagtagaaa |
| 5821 | gttagattgc tcaactacaa ctcctctaga actctatagc tctgacatac agattcacac |
| 5881 | tctcctctat ttgctaagta tgtaaagaat gttttctttt aaaatgttct cttttgagaa |
| 5941 | caactgctta tttgttataa aagcatttgg ttaaaatgat gtcatcataa aaaacagtgg |
| 6001 | ctttgtttca atacatattt ttgagatgat tatctagaag ccagattaat aaaatcagct |
| 6061 | tgtgaccttg ctaagcatat aaactggaaa ttcagataca ttcaaaatta tgggttcatt |
| 6121 | taaaagtgtt ctaccttttg ggtatgagac taatatcact aattcctcaa tagttatcat |
| 6181 | ggctctatct taattaatta gaaaatatgt gtgtttaatt ctttgagaat taaaatagag |
| 6241 | aatattaaca gagggttaaa aactgcttca actccaataa gataaaggaa gctcaaaatc |
| 6301 | tatgagctga gtgttcaatt agctttgcct actgagttca attttatgtc aatacaacag |
| 6361 | tggatcagac agtacgactt tgaactggtg aatgtaaaca attgtttttc acctaagctg |
| 6421 | ctttggaaga actgatgctt gctgctaact aaagttttgg atgtatcgat ttagagaacc |
| 6481 | aattaatacc tgcaaaataa agcatactgt ggtacttctg tttgatctag tatgtgtgat |
| 6541 | tttagattga tggattaaaa attaataaag atcatacatt ccataccaaa aaaaaaaaaa |
| 6601 | aaa |

SEQ ID NO: 12 GenBank accession number: NM_002294, incorporated herein by reference.

In embodiments, the composition further comprises a latency reversal agent (LRA).

The amino acid sequence of the IL-16/Lamp2b fusion protein is depicted below. The IL-16 sequence is bold, and the Lampb2 sequence is underlined and the Human influenza hemagglutinin (HA)-Tag is highlighted in grey:

(SEQ ID NO: 13)
MVCFRLFPVPGSGLVLVCLVLGAVRSYAGNSTMGSGTIVIR
RKSLQSKETTAAGDSGSGSGSGGSSLELNLTDSENATCLYA
KWQMNFTVRYETTNKTYKTVTISDHGTVTYNGSICGDDQN
GPKIAVQFGPGFSWIANFTKAASTYSIDSVSFSYNTGDNTTF
PDAEDKGILTVDELLAIRIPLNDLFRCNSLSTLEKNDVVQHY
WDVLVQAFVQNGTVSTNEFLCDKDKTSTVAPTIHTTVPSPT
TTPTPKEKPEAGTYSVNNGNDTCLLATMGLQLNITQDKVAS
VININPNTTHSTGSCRSHTALLRLNSSTIKYLDFVFAVKNEN
RFYLKEVNISMYLVNGSVFSIANNNLSYWDAPLGSSYMCNK
EQTVSVSGAFQINTFDLRVQPFNVTQGKYSTAQECSLDDDTI

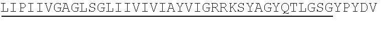

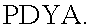

The nucleic acid sequence encoding the IL-16/Lamp2b fusion protein is depicted below. The IL-16 sequence is bold, and the Lampb2 sequence is underlined and the HA-Tag is highlighted in grey:

(SEQ ID NO: 14)
atggtgtgcttccgcctcttcccggttccgggctcagggctcgttctggtctgcctagtcctgggagctgtgcggtcttatgcaggtaact
cgactatgggcagtggaacgattgtcatcaggagaaaaagcctccagtccaaggaaaccacagctgctggagactccggcag
tggatctggatccggtggctcgagtttggaacttaatttgacagattcagaaaatgccacttgcctttatgcaaaatggcagatgaatttca
cagttcgctatgaaactacaaataaaacttataaaactgtaaccatttcagaccatggcactgtgacatataatggaagcatttgtgggga
tgatcagaatggtcccaaaatagcagtgcagttcggacctggcttttcctggattgcgaattttaccaaggcagcatctacttattcaattg
acagcgtctcattttcctacaacactggtgataacacaacatttcctgatgctgaagataaaggaattcttactgttgatgaacttttggcca
tcagaattccattgaatgacctttttagatgcaatagtttatcaactttggaaaagaatgatgttgtccaacactactgggatgttcttgtaca
agcttttgtccaaaatggcacagtgagcacaaatgagttcctgtgtgataaagacaaaacttcaacagtggcacccaccatacacacca
ctgtgccatctcctactacaacacctactccaaaggaaaaaccagaagctggaacctattcagttaataatggcaatgatacttgcctgct
ggctaccatggggctgcagctgaacatcactcaggataaggttgcttcagttattaacatcaaccccaatacaactcactccacaggca
gctgccgttctcacactgctctacttagactcaatagcagcactattaagtatctagactttgtctttgctgtgaaaaatgaaaaccgatttta
tctgaaggaagtgaacatcagcatgtatttggttaatggctccgttttcagcattgcaaataacaatctcagctactgggatgcccccctg
ggaagttcttatatgtgcaacaaagagcagactgtttcagtgtctggagcatttcagataaatacctttgatctaagggttcagcctttcaat
gtgacacaaggaaagtattctacagcccaagagtgttcgctggatgatgacaccattctaatcccaattatagttggtgctggtctttcag

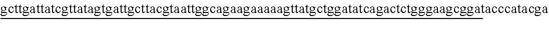

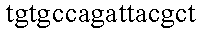

The term “latency reversing drug combination”, “combination therapy”, or “latency reversing agents” includes but not limited to combinations of the following drugs: Protein Kinase C (PKC) agonists, bromo and external (BET) bromodomain inhibitors, histone deacetylase (HDAC) inhibitors, and acetaldehyde dehydrogenase inhibitor, and activator of nuclear factor kappa-light chain-enhancer of activated B cells (NF-κB) and the AKT pathway. In certain embodiments, the PKC agonist is biyostatin-1, prostratin, ingenol-3-angelate, ingenol mimic, or DAG mimic

In certain embodiments, the HDAC inhibitor is selected from the group consisting of vorinostat, panobinostat, and romidepsin. In other embodiments, the HDAC inhibitor is selected from 4-phenylbutyrohydroxamic acid, Acetyldinaline, APHA, Apicidin, AR-42, Belinostat, CUDC-101, CUDC-907, Dacinostat, Depudecin, Droxinostat, Entinostat, Givinostat, HC-Toxin, ITF-2357, JNJ-26481585, KD 5170, LAQ-824, LMK235, M344, MC1568, MGCD-0103, Mocetinostat, NCH 51, Niltubacin, NSC3852, Oxamflatin, Panobinostat, PCI-24781, PCI-34051, Pracinostat, Pyroxamide, Resminostat, RG2833, RGFP966, Rocilinostat, Romidepsin, SBHA, Scriptaid, Suberohydroxamic acid, Tacedinaline, TC-H 106, TCS HDAC6 20b, Tacedinaline, TMP269, Trichostatin A, Tubacin, Tubastatin A, Valproic acid, or Vorinostat.

In certain embodiments, the bromodomain inhibitor is JQ1. In other embodiments, the BET inhibitor is selected from CPI 203, 1-BET151, 1-BET762, JQ1, MS417, MS436, OTX-015, PFi-1, or RVX-208. In certain embodiments, the latency reversing drug combinations comprise acetaldehyde dehydrogenase inhibitor, activator of F-κB and the AKT pathway with HDAC inhibitors. In certain embodiments, the latency reversing drug combinations comprise PKC agonists with bromodomain inhibitors. In certain embodiments, the latency reversing drug combinations comprise disulfiram with vorinostate. In certain embodiments, the latency reversing drug combinations comprise disulfiram with panobinostat. In certain embodiments, the latency reversing drug combinations comprise disulfiram with romidepsin. In certain embodiments, the latency reversing drug combinations comprise biyostatin-1 with JQ1. In certain embodiments, the latency reversing drug combinations comprise prostratin with JQl.

In embodiments, the latency reversal agent comprises an HIV Tat polypeptide. The Human Immunodeficiency Virus (HIV) trans-activator of transcription (Tat) is a variable RNA binding peptide of 86 to 110 amino acids in length that is encoded on two separate exons of the HIV genome. In examples, the variant comprises 86 amino acids, and in other examples, the variant comprises 101 amino acids, or 110 amino acids.

In embodiments, the Tat protein sequence comprises 110 amino acids:

(SEQ ID NO: 15)
MEPVDPNLEPWKHPGSQPRTACNNCYCKKCCFHCYACFTRKGLGISYGR
KKRRQRRRAPQDSQTHQASLSKQPASQSRGDPTGPTESKKKVERETETD
PFDYPYDVPDYAA

In embodiments, the Tat cDNA sequence comprises:

(SEQ ID NO: 16; start codon is underlined)
ATGGAGCCAGTAGATCCTAATCTAGAGCCCTGGAAGCATCCAGGAAGTCA
GCCTAGGACTGCTTGTAACAATTGCTATTGTAAAAAGTGTTGCTTTCATT
GCTACGCGTGTTTCACAAGAAAAGGCTTAGGCATCTCCTATGGCAGGAAG
AAGCGGAGACAGCGACGAAGAGCTCCTCAGGACAGTCAGACTCATCAAGC
TTCTCTATCAAAGCAACCCGCCTCCCAGTCCCGAGGGGACCCGACAGGGC
CCACGGAATCGAAGAAGAAGGTGGAGAGAGAGACAGAGACAGATCCGTTC
GATTACCCATACGATGTTCCAGATTACGCTGCC

In embodiments, the Tat protein sequence comprises 101 amino acids:

(SEQ ID NO: 17)
M E P V D P N L E P W K H P G S Q P R T A C N N C
Y C K K C C F H C Y A C F T R K G L G I S Y G R K
K R R Q R R R A P Q D S Q T H Q A S L S K Q P A S
Q S R G D P T G P T E S K K K V E R E T E T D P F
D

In embodiments, the Tat cDNA sequence comprises:

(SEQ ID NO: 18)
ATGGAGCCAGTAGATCCTAATCTAGAGCCCTGGAAGCATCCAGGAAGTCA
GCCTAGGACTGCTTGTAACAATTGCTATTGTAAAAAGTGTTGCTTTCATT
GCTACGCGTGTTTCACAAGAAAAGGCTTAGGCATCTCCTATGGCAGGAAG
AAGCGGAGACAGCGACGAAGAGCTCCTCAGGACAGTCAGACTCATCAAGC
TTCTCTATCAAAGCAACCCGCCTCCCAGTCCCGAGGGGACCCGACAGGGC
CCACGGAATCGAAGAAGAAGGTGGAGAGAGAGACAGAGACAGATCCGTTC
GAT

In embodiments, the Tat amino acid sequence comprises 86 amino acids:

(SEQ ID NO: 19)
M E P V D P N L E P W K H P G S Q P R T A C N N C
Y C K K C C F H C Y A C F T R K G L G I S Y G R K
K R R Q R R R A P Q D S Q T H Q A S L S K Q P A S
Q S R G D P T G P T E

In embodiments, the Tat cDNA sequence comprises:

(SEQ ID NO: 20)
ATGGAGCCAGTAGATCCTAATCTAGAGCCCTGGAAGCATCCAGGAAGTC
AGCCTAGGACTGCTTGTAACAATTGCTATTGTAAAAAGTGTTGCTTTCA
TTGCTACGCGTGTTTCACAAGAAAAGGCTTAGGCATCTCCTATGGCAGG
AAGAAGCGGAGACAGCGACGAAGAGCTCCTCAGGACAGTCAGACTCATC
AAGCTTCTCTATCAAAGCAACCCGCCTCCCAGTCCCGAGGGGACCCGAC
AGGGCCCACGGAA

Tat is highly conserved among all human lentiviruses and is essential for viral replication. When lentivirus Tat binds to the TAR (trans-activation responsive) RNA region, transcription (conversion of viral RNA to DNA then to messenger RNA) levels increase significantly. It has been demonstrated that Tat increases viral RNA transcription and it has been proposed that Tat may initiate apoptosis (programmed cell death) in T4 cells and macrophages (a key part of the body's immune surveillance system for HIV infection) and possibly stimulates the over production of alpha interferon (α-interferon is a well-established immunosuppressive cytokine).

In embodiments, the TAT peptide is derived from the transactivator of transcription (TAT) of human immunodeficiency virus and is a Cell-penetrating peptides. Cell-penetrating peptides (CPPs) have been used to overcome the lipophilic barrier of the cellular membranes and deliver large molecules and even small particles inside the cell for their biological actions. CPPs are being used to deliver inside cell a large variety of cargoes such as proteins, DNA, antibodies, contrast (imaging) agents, toxins, and nanoparticle drug carriers including liposomes.

In embodiments, the amino terminal portion of Tat includes a short peptide region from a nuclear transcription factor (TF) typically flanked by proline residues, and comprises the amino acid sequence: (MGCINSKRKD SEQ ID NO: 29), which leads Tat to the cell membrane.

This region determines, at least in part, how stimulatory or suppressive the Tat polypeptide is for cells of the immune system, particularly innate immune cells such as dendritic cells (DC) and macrophages (antigen-presenting cells or APCs).

In embodiments, the Tat peptide comprises the amino acid sequence:

(SEQ ID NO: 15)
MEPVDPNLEPWKHPGSQPRTACNNCYCKKCCFHCYACFTRKGLGISYGR
KKRRQRRRAPQDSQTHQASLSKQPASQSRGDPTGPTESKKKVERETETD
PFDYPYDVPDYAA

In embodiments, the Tat peptide comprises the amino acid sequence:

(SEQ ID NO: 17)
M E P V D P N L E P W K H P G S Q P R T A C N N C
Y C K K C C F H C Y A C F T R K G L G I S Y G R K
K R R Q R R R A P Q D S Q T H Q A S L S K Q P A S
Q S R G D P T G P T E S K K K V E R E T E T D P F
D

In embodiments, the Tat peptide comprises the amino acid sequence: GRKKRRQRRRAP (SEQ ID NO: 6). In embodiments, the Tat peptide comprises the amino acid sequence: RKKRRQRRR (SEQ ID NO: 28).

In embodiments, the composition comprising the exosome comprises a nuclear localization signal (NLS) c-myc (PAAKRVKLD SEQ ID NO: 2), nucleoplasmin (AVKRPAATKKAGQAKKKKLD SEQ ID NO: 3), EGL-13 (MSRRRKANPTKLSENAKKLAKEVEN SEQ ID NO: 4), TUS-protein (KLKIKRPVK SEQ ID NO: 5), HIV-1 Tat (GRKKRRQRRRAP SEQ ID NO: 6), or the HIV-1 Tat (RKKRRQRRR SEQ ID NO: 28). In embodiments, the nuclear localization signal comprises myc. The NLS is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS.

In embodiments, the NLS comprises c-myc. In embodiments, the c-myc comprises the amino acid sequence: PAAKRVKLD (SEQ ID NO: 2).

Exemplary NLS sequences are provided in Table 1 below:

TABLE 1
NLS motifs

| | | SEQ ID |
|---|---|---|
| NLS^{a} | Protein | NO: |
| RRMKWKK | PDX-1 | 30 |
| RVHPYQR | QKI-5 | 31 |
| KRPACTLKPECVQQLLVCSQEAKK | HCDA | 32 |
| PKKKRKV | SV40 LrgT | 33 |
| GKKRSKA | H2B | 34 |
| KAKRQR | v-Rel | 35 |
| RGRRRRQR | Amida | 36 |
| RKRRR | Amida | 37 |
| PPVKRERTS | RanBP3 | 38 |
| PYLNKRKGKP | Pho4p | 39 |
| CYGSKNTGAKKRKIDDA | DNAhelicaseQ1 | 40 |
| KKKKRKREK | LEF-1 | 41 |
| KKKRRSREK | TCF-1 | 42 |
| RKRIREDRKx{18}RKRKR | TCPTP | 43 |
| RRERx{4}RPRKIPR | BDV-P | 44 |
| KKKKKEEEGEGKKK | Act/inhβA | 45 |
| PRPRKIPR | BDV-P | 46 |
| PPRIYPQLPSAPT | BDV-P | 47 |
| KDCVINKHHRNRCQYCRLQR | TR2 | 48 |
| Krx{9}KTKK | THOV NP | 49 |
| APKRKSGVSKC | PolyomaVP1 | 50 |
| RQARRNRRRRWR | HIV-1 Rev | 51 |
| MPKTRRRPRRSQRKRPPT | Rex | 52 |
| KRPMNAFIVWSRDQRRK | SRY | 53 |
| PRRRK | SRY | 54 |
| KRPMNAFMVWAQAARRK | SOX9 | 55 |
| PRRRK | SOX9 | 56 |
| [KAR]TPIQKHWRPTVLTEGP | AS Vintegrase | 57 |
| PVKIRIETGEWE[KA] | | |
| PPRKKRTVV | NS5A | 58 |
| YKRPCKRSFIRFI | DNAse EBV | 59 |
| LKDVRKRKLGPGH | DNAse EBV | 60 |
| KRPRP | AdenovE la | 61 |
| RKRKKKRAAEDDEDDDVDTKKQK | YstDNApolα/ | 62 |
| | HProTα | |
| GRKRKKRT | Tstl/Oct6 | 63 |
| KKKQKK | 20S proteasome | 64 |
| REKKEKEQKEKCA | Prot.Hsc9 | 65 |
| LEKKVKKKBDWCA | Prot.Hsci | 66 |
| TEKK[QG]KSILYDCA | Prot.Hsc3 | 67 |
| SDKKVRSRLIECA | Ta α | 68 |
| LKRKLQR | Pax-QNR | 69 |
| RRKGKEK | Hunt.Dis.prot | 70 |
| CKRKTTNADRRKA | MyoD | 71 |
| VNEAFETLKRC | MyoD | 72 |
| MPTEERVRKRKESNRESARRSRYRK | Opaque2 | 73 |
| AAHLK | | |
| KVNSRKRRKEVPGPNGATEED | CTP | 74 |
| PRRGPR | HCV | 75 |
| PRGRRQPIPKARQP | HCV | 76 |
| KRSAEGGNPPKPLKKLR | P1l0RB1 | 77 |
| KRKx{11}KKKSKK | Hpoly(ADP)poly | 78 |
| EYLSRKGKLEL | VirD2-Nterm | 79 |
| PKRPRDRHDGELGGRKRARG | VirD2-Cterm | 80 |
| KRPAATKKAGQAKKKK | Nuclopasmin | 81 |
| KRKKEMANKSAPEAKKKK | Nucleolin | 82 |
| RKRAFHGDDPFGEGPPDKK | ICP-8 | 83 |
| GGGx{3}KNRRx{6}RGGRN | Nab2 | 84 |
| YNNQSSNFGPMKGGN | M9 | 85 |
| PAAKRVKLD | LscMyc | 86 |
| KRPAEDMEEEQAFKRSR | HumKprotein | 87 |
| SxGTKRSYxxM | FluA | 88 |
| MNKIPIKDLLNPG | Matα | 89 |
| PKKARED | Polyoma Lrg-T | 90 |
| VSRKRPR | Polyoma Lrg-T | 91 |
| APTKRKGS | S V40 VP1 | 92 |
| PNKKKRK | S V40 VP2 | 93 |
| EEDGPQKKKRRL | Polyoma VP2 | 94 |
| PLLKKIKQ | c-myb | 95 |
| PPQKKIKS | N-myc | 96 |
| PQPKKKP | P53 | 97 |
| SKRVAKRKL | c-erb-A | 98 |
| IKYFKKFPKD | Yeast SKI3 | 99 |
| KTRKHRG | L29 | 100 |
| KHRKHPG | L29 | 101 |
| PQSRKKLR | Max | 102 |
| HRKYEAPRHx{6}PRKR | L3 | 103 |
| KKEKKKSKK | Dyskerin | 104 |

^{a}Motif, notation: [K, R], K or R, i.e., any of the two amino acids at that position, x: any amino acid; x{9}, 9 times x; and x{7, 9}, at least 7, at most 9 times x.

Additional NLS sequences may be used and are identified in Cokol, M. et al. 2000 EMBO Report, 1(5): 411-415, incorporated herein by reference in its entirety.

### Methods for Promoting Viral Transcription

Provided herein are methods for promoting viral transcription in a cell. In embodiments, the method comprises contacting an HIV-infected CD4+T cell with a composition. In embodiments, the composition comprises an exosome, which comprises a surface-exposed interleukin 16 (IL-16) polypeptide.

The methods described herein increase viral transcription. For example, the viral transcription increases by about 50% to about 1200% compared to the level of viral transcription without contacting an HIV-infected CD4+T cell with the composition described herein (e.g., the composition comprising an exosome wherein the exosome comprises a surface-exposed interleukin-16 (IL-16) polypeptide). The level of viral transcription increases by about 50% to about 1000%, by about 50% to about 900%, by about 50% to about 800%, by about 50% to about 700%, by about 50% to about 600%, by about 50% to about 500%, by about 50% to about 400%, by about 50% to about 300%, by about 50% to about 200%, or by about 50% to about 100% compared to the level of viral transcription without contacting an HIV-infected CD4+T cell with the composition described herein (e.g., the composition comprising an exosome wherein the exosome comprises a surface-exposed interleukin-16 (IL-16) polypeptide).

In some examples, the cargo comprises a peptide such as a Tat polypeptide. The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

In embodiments, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

In embodiments, the cargo comprises a protein therapeutic. In embodiments, protein therapeutics can include peptides, enzymes, structural proteins, receptors, cellular proteins, or circulating proteins, or fragments thereof.

In embodiments, the cargo comprises a therapeutic agent. A therapeutic agent, e.g., a drug, or an active agent, can mean any compound useful for therapeutic or diagnostic purposes, the term can be understood to mean any compound that is administered to a patient for the treatment of a condition. Accordingly, a therapeutic agent can include, proteins, peptides, antibodies, antibody fragments, and small molecules.

### Method of Preparing Composition

Provided herein, are methods for preparing an exosome comprising a surface-exposed interleukin-16 (IL-16) polypeptide. The method comprises culturing cells in a medium. The cells release the exosomes by secretion into the medium, collecting the supernatant of medium, fractionating the supernatant comprising the exosomes, and isolating the exosomes. For example, the fractionating can include separation methods comprising centrifugation (e.g., density centrifugation) or immunological methods (e.g., antibody beads). Additional methods include ultracentrifugation, ultrafiltration, polymer-based reagents, size exclusion chromatography, density gradient separation, and immunoaffinity capture. Fractionating methods can be found at Lane R. et al., 2017 Methods in Molecular Biology, vol. 1660: 111-130, incorporated herein by reference in its entirety.

The cells comprise, but are not limited to eukaryotic cells.

Exosomes are produced by many different types of cells including immune cells such as B lymphocytes, T lymphocytes, dendritic cells (DCs) and mast cells. Exosomes are also produced, for example, by glioma cells, platelets, reticulocytes, neurons, intestinal epithelial cells, tumor cells, HELA cells, human embryonic kidney cells (HEK cells), B2M17 cells, Bend3 cells, primary bone marrow-derived dendritic cells, BV-2 microglia cells and EURO2A cells. Exosomes for use in accordance with the present application can be derived from any suitable cell, including, but not limited to the cells identified above.

In embodiments, the cells that release the exosome by secretion into the medium comprise a protein of interest. In embodiments, the protein of interest comprises a viral protein. In embodiments, the viral protein comprises HIV Tat.

### Method of Treating

Provided herein are methods of treating a patient comprising a viral infection.

In an embodiment, the viral infection is caused by a virus called human immunodeficiency virus (HIV). In an embodiment, the viral infection is caused by HIV, e.g., HIV-1.

The method comprises administering to the patient an effective amount of a pharmaceutical composition comprising an exosome, wherein the exosome comprises a surface-exposed interleukin-16 (IL-16) polypeptide.

In embodiments, the patient is administered the composition intravenously. In embodiments, engineered exosomes may be administered by intravenous, intracutaneous, intraperitoneal, intramuscular, intra-articular, intraarterial, intrasynovial, intrasternal, intrathecal, intralesional and intracranial injection or infusion techniques. In embodiments, viral vectors described herein may be administered by intravenous administration, intramuscular administration, subcutaneous administration, or intrathecal administration.

The composition may be administered in a range from about 1×10⁹ to about 2×10⁹ genomic copies/mouse. Alternatively, the composition may be administered in an amount equivalent to a protein standard. The conversion of animal doses to human equivalent doses based on body surface area in shown in Table 4 below (based on FDA Guidance, “Guidance for Industry Estimating the Maximum Safe Starting Dose in Initial Clinical Trials for Therapeutics in Adult Healthy Volunteers,” U.S. Department of Health and Human Services Food and Drug Administration Center for Drug Evaluation and Research (CDER) July 2005 Pharmacology and Toxicology, incorporated herein by refernce in its entirety).

TABLE 4
The conversion of animal doses to human equivalent doses
Table 1
Conversion of Animal Doses to Human
Equivalent Doses Based on Body Surface Area

| | To Convert | | |
|---|---|---|---|
| | Animal Dose in | To Convert Animal Dose in mg/kg | |
| | mg/kg to Dose in | to HED^{a} in mg/kg, Either: | |
| | mg/m², Multiply | Divide | Multiply |
| Species | by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

In accordance with the methods described herein, a “subject in need of” is a subject having a viral disease, or a subject having an increased risk of developing a viral disease, relative to the population at large. The subject in need thereof can be one that is “non-responsive” or “refractory” to a currently available therapy for the viral disease. In this context, the terms “non-responsive” and “refractory” refer to the subject's response to therapy as not clinically adequate to relieve one or more symptoms associated with the viral infection. In one aspect of the methods described here, the subject in need thereof is a subject having a viral disease caused by an HIV virus who is refractory to standard therapy. The patient (e.g., subject) comprises a human. The effective amount is an amount effective to promote viral transcription. The therapeutically effective amount is an amount effective to achieve one or more of the following: promote viral transcription, ameliorate one or more symptoms associated with viral infection of the subject, and reduce the severity of one or more symptoms associated with viral infection of the subject. The therapeutically effective amount is in an amount to enhance host defense against viral pathogens. In an embodiment, the therapeutically effective amount is in an amount that is synergistic to promote host defense against viral pathogens. The effective amount is from about 0.01 ng to about 10,000 ng of the composition. The composition comprises a concentration containing about, at least about, or at most about 0.01, 1.0, 10.0, 100, 200, 300, 400, 500, 600, 700, 800, 900, 1000, 2000, 3000, 4000, 5000, 6000, 7000, 8000, 9000, or 10000 nanograms (ng), of exosomes, or any range derivable therein. The above numerical values may also be the dosage that is administered to the patient based on the patient's weight, expressed as ng/kg, mg/kg, or g/kg, and any range derivable from those values. The composition may have a concentration of exosomes that are 0.01, 1.0, 10.0, 100, 200, 300, 400, 500, 600, 700, 800, 900, or 1000 ng/ml, or any range derivable therein. The effective amount is from about 0.01 ng/mL to about 10,000 ng/mL of the composition.

In embodiments, the composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein.

In embodiments, the composition may be administered once daily, twice daily, three times daily, four times daily, five times daily, or six times daily (or any range derivable therein) and/or as needed to the patient.

In embodiments, the composition may be administered every 2, 4, 6, 8, 12 or 24 hours (or any range derivable therein) to or by the patient. In some embodiments, the patient is administered the composition for a certain period of time or with a certain number of doses.

In embodiments, the composition is administered in an amount of 0.001 to 1000 mg/day. In embodiments, the composition is administered in a range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

In embodiments, methods comprising combination therapy are provided. As used herein, “combination therapy” or “co-therapy” includes the administration of a therapeutically effective amount of a pharmaceutical composition comprising an exosome, wherein the exosome comprises a surface-exposed interleukin-16 (IL-16) polypeptide, with at least one additional active agent, as part of a specific treatment regimen intended to provide a beneficial effect from the co-action of the active agents in the regimen, e.g. anti-retroviral drug such as truvada (Emtricitabine/tenofovir).

The at least one additional active agent may be a therapeutic agent, for example an anti-viral agent, or a non-therapeutic agent, and combinations thereof. With respect to therapeutic agents, the beneficial effect of the combination includes, but is not limited to, pharmacokinetic or pharmacodynamic co-action resulting from the combination of therapeutically active compounds. With respect to non-therapeutic agents, the beneficial effect of the combination may relate to the mitigation of toxicity, side effect, or adverse event associated with a therapeutically active agent in the combination.

For example, the therapeutic agent is selected from an anti-viral agent, an anti-viral vaccine, a nucleotide analogue, a cytokine (e.g., an interferon), and an immunoglobulin, and combinations thereof. In an embodiment, the one additional agent is an anti-viral agent. Non-limiting examples of anti-viral agents that may be used in combination with a composition comprising an exosome, wherein the exosome comprises a surface-exposed interleukin-16 (IL-16) polypeptide, as described herein include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In examples, the therapeutic agent is a combination drug. For example, the combination drug comprises Stribild (Elvitegravir/cobicistat/emtricitabine/tenofovir by Gilead), Atriplia (Efavirenz/emtricitabine/tenofovir by Gilead), Ziagen (abacavir by Merck), Reyataz (atazanavir by Bristol-Myers Squibb), Norvir (ritonavir by Abbvie), Truvada (Emtricitabine/tenofovir by Gilead), Isentess (Raltegravir by Merck), Sustiva (efavirenz by Bristol-Myers Squibb), 3TC (Lamivudine), Triumeq (Abacavir/dolutegravir/lamivudine by GlaxoSmithKline/ViiV Healthcare), Epzicom (Abacavir/lamivudine by Kivexa and ViiV Healthcare), Prezista (Darunavir by Janssen).

Pharmaceutical Compositions and Formulations

The present invention provides pharmaceutical compositions comprising an effective amount of a composition comprising an exosome comprising a surface-exposed interleukin 16 (IL-16) polypeptide and at least one pharmaceutically acceptable excipient or carrier, wherein the effective amount is as described above in connection with the methods of the invention. In an embodiment, the exosome comprises a lysosomal-associated membrane protein (lamp)/IL-16 fusion protein.

In one embodiment, the composition comprising an exosome comprising a surface-exposed interleukin 16 (IL-16) polypeptide is further combined with at least one additional therapeutic agent in a single dosage form. In one embodiment, the at least one additional therapeutic agent is selected from an anti-viral agent (as described above), an anti-viral vaccine, a nucleotide analogue, a cytokine (e.g., an interferon), and an immunoglobulin, and combinations thereof.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an ingredient that is useful in preparing a pharmaceutical composition and is inactive. Such an excipient or carrier ingredient is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating viral infections are described.

A dose may be provided in unit dosage form. For example, the unit dosage form can comprise 1 nanogram to 2 milligrams, or 0 1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, intravenous, intramuscular, pulmonary, inhalation, intranasal, oral, buccal, sublingual, parenteral, subcutaneous, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples.

Kits

Provided herein are kits comprising one or more reagents for preparing an exosome comprising a surface-exposed interleukin 16 (IL-16). In embodiments, the exosome further comprises a latency reversal agent.

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

The provided examples illustrate different components and methodology useful in practicing the present disclosure. The examples do not limit the claimed disclosure. Based on the present disclosure the skilled artisan can identify and employ other components and methodology useful for practicing the present disclosure.

### Example 1

### Engineered HIV-1 Tat Expression in Exosomes

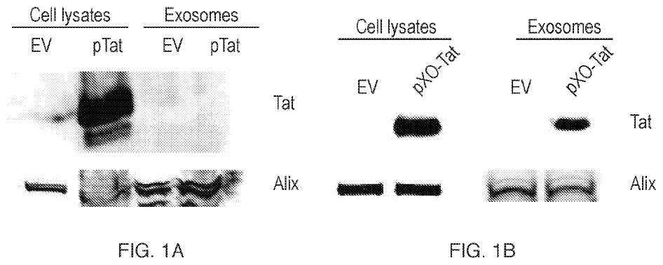

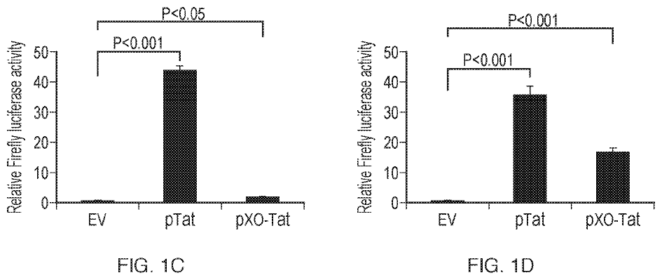

As seen in FIG. 1A, transfection of a Tat expression vector (pTat) into HEK293T cells led to robust levels of intracellular Tat but no detectable expression in released exosomes of 30-150 nm diameter. The expression vector was modified to include a previously characterized peptide sequence that when placed upstream of coding sequence, directs proteins to the interior exosomal membrane (de Gassart, A. et al. Blood. 15;102, 4336-4344 (2003)). The modified vector (pXO-Tat) was transfected into HEK293T cells and led to robust expression of Tat in released exosomes (FIG. 1B).

The function of the modified Tat construct was tested in TZM-bl cells, an indicator cell line which enables quantitative analysis of HIV-1 promoter LTR activation using either luciferase or β-gal as a reporter (Folks, T M. et al. Science. 238, 800-802 (1987)). Transfection of pXO-Tat into TZM-bl cells significantly increased HIV-1 promoter LTR activation, but at levels far less than that of wild-type pTat (FIG. 1C). Given that the biological activity of Tat derives from its nuclear localization, the placement of a membrane localization signal allowed shuttling between plasma membrane and nuclear compartments reducing Tat protein levels in the latter and thereby HIV-1 trans activating activity.

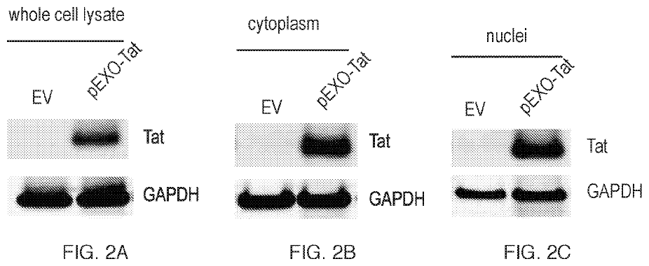

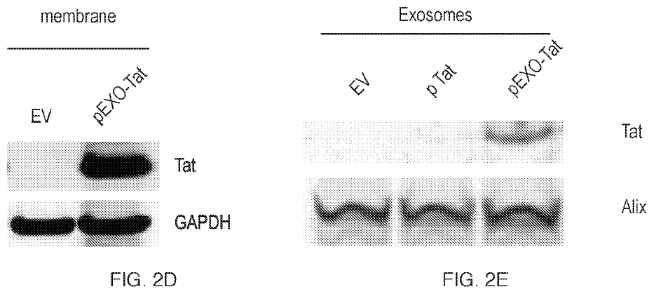

To retain exosomal localization yet increase nuclear presence, a C-myc Nuclear Localization Signal (NLS) was fused to the C-terminus of Tat. The biological activity of this new construct (pEXO-Tat) was quantified using the TZM-bl reporter system with pEXO-Tat activating the HIV-1 promoter LTR 9-fold more than pXO-Tat with about 50% potency of wild-type pTat (FIG. 1D). The subcellular distribution of EXO-Tat was profiled by Western blot. As seen in FIG. 2A-2E, Tat protein could be readily detected not only in cytoplasmic, nuclear and membrane fractions but also in a heterogeneous population of released extracellular vesicles.

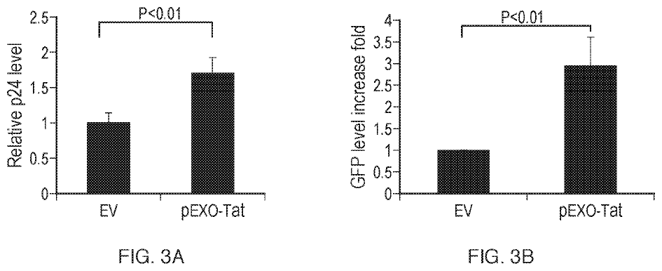

To further verify that the manipulation of the N- and C-terminal sequences of Tat had no impact on its transactivating activity, U1 cells, a pro-monocytic cell line engineered to harbor integrated HIV-120 was used. U1 cells have minimal levels of baseline viral expression that increase after treatment with agents that activate the HIV-1 LTR. Transfection of pEXO-Tat into U1 cells led to an increase in virion release as quantified by serial p24 protein measurement in cellular supernatants (FIG. 3A). These data were duplicated using other in vitro models of HIV-1 latency including the J-Lat GFP (clone A72) in which viral promoter (LTR) activation is tracked by GFP expression (Jordan, A. et al. EMBO J. 22, 1868-1877 (2003)). As seen in FIG. 3B, transfection of pEXO-Tat into J-Lat GFP (clone A72) cells led to 3-fold increase in GFP expression compared to relevant control.

### Example 2

### Exosomal Tat Activated Latent HIV-1 in Primary Resting CD4+T Lymphocytes

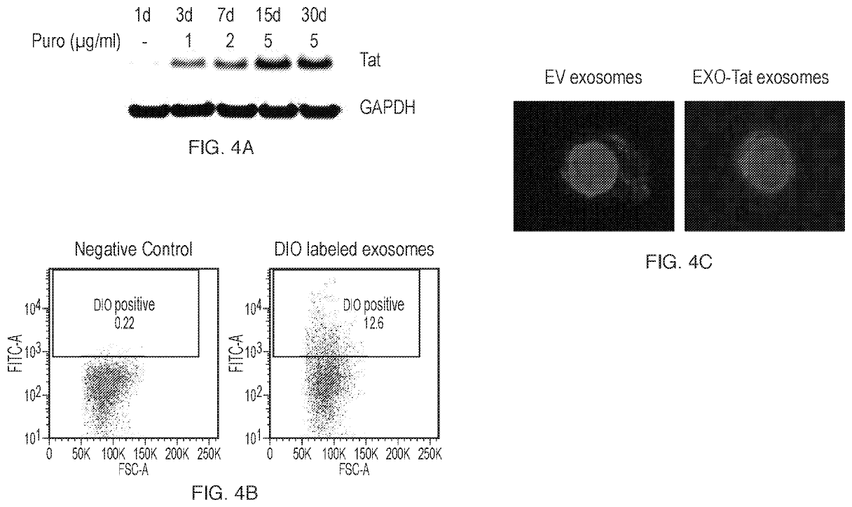

Transient transfection was used to generate EXO-Tat exosomes and each transfection led to limited numbers of exosomes secreted in cellular supernatants. To facilitate down-stream experiments, the scale of production was increased by transduction of HEK293T cells with a lentiviral construct harboring the EXO-Tat expression system. Cells were placed under drug selection and serial sampling of cell lysates revealed robust Tat production after 15 days of drug selection (FIG. 4A). Exosomes were stained with a highly lipophilic dye (DiO) and flow cytometry was used to quantify exosomal cellular uptake kinetics. As seen in FIG. 4B, ˜13% of CD4+T lymphocytes were DiO+ in agreement with results obtained using immunofluorescent staining (FIG. 4C).

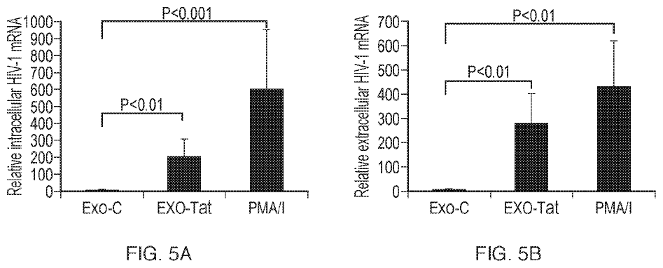

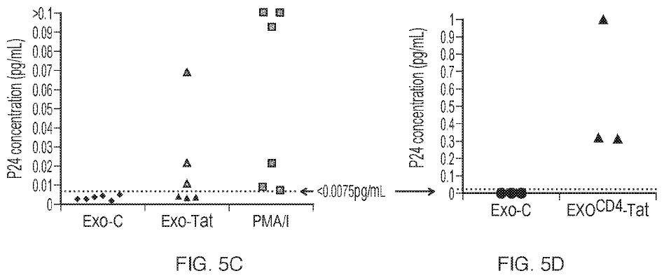

Primary, resting [HLA-DR (−), CD25 (−), CD69 (−)] CD4+T lymphocytes isolated from five HIV-1 infected individuals successfully treated with antiretroviral drugs with prolonged periods of viral suppression (Patient IDs: #211, #219, #232, #111 and #207 in Table 3) were used. Highly purified preparations of resting (r) CD4+ cells (˜2×10⁶) were placed in culture and treated with control exosomes or exosomes harboring EXO-Tat (1.8×10⁹ exosomes or 46.8 μg total protein) for 96 hours. As seen in FIG. 5A and 5B, EXO-Tat exosomal treatment led to the expression of HIV-1 unspliced RNA in all seven individuals. Levels of intracellular HIV-1 RNA were below the level of detection in rCD4+ lymphocytes treated with control exosomes (Exo-C) in every case examined. Cellular RNA was subjected to Tat/rev Induced Limiting Dilution Assay (TILDA) (Procopio, F A. et al. EBioMedicine. 2, 874-883 (2015)). This PCR reaction measures inducible multiply-spliced HIV RNA (msRNA), the presence of which is thought to correlate with replication competent HIV-1. Four (#211, #219, #211 and #207) out of 5 patient samples were positive for tat/rev msRNA.

While nucleic acid based assays can quantify the transcription of integrated HIV-1, the mere presence of transcription does not always correlate with cellular production and release of infectious virions. In the majority of cells infected by HIV-1, integrated virus is defective due to the error prone nature of the viral enzyme reverse transcriptase that converts incoming virion RNA into DNA. To confirm that EXO-Tat exosomes reactivated latent HIV-1 to produce replication-competent virus, isolated rCD4+T cells were isolated from another 6 ART-treated individuals (Patient IDs: #112, #204, #225, #108, #223 and #109 in Table 3), and treated with control exosomes (Exo-C), EXO-Tat exosomes or the global immune activator PMA/I for 4 days. The respective supernatants were subsequently co-cultured with MOLT-4 cells and viral p24 antigen in cell culture supernatants was quantified by ELISA. EXO-Tat exosomes induced p24 production in 3 out of 6 patient samples (FIG. 5C).

### Example 3

### Exosomes Were Engineered to Target CD4+ Cells Which Improved Latency rRversal Activity of EXO-Tat

Limiting dilution assays revealed that ≤1/10⁶ rCD4+T lymphocytes were infected with replication competent HIV-17 Laird, G M. et al. Methods Mol Biol. 1354, 239-253 (2016), and Bullen, C K. et al. Nat Med. 20, 425-429 (2014)). Exosomes made to specifically target CD4+ expressing cells were more potent in terms of activating latent HIV-1. Exosomes were modified by expressing EXO-Tat with a construct encoding an Interleukin (IL)-16 C-terminal 20 amino acid domain fused to the N-terminus of lysosome-associated membrane protein 2 variant b (Lamp2b).

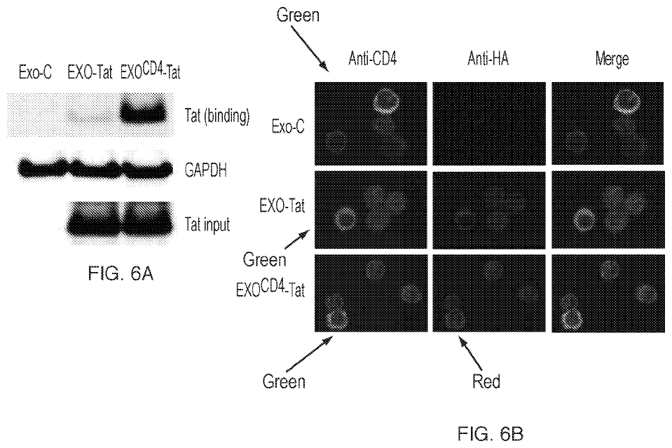

IL-16 is a natural ligand for the CD4 receptor with the minimal peptide RRKS (SEQ ID NO: 1) within the C-terminus of IL-16 being critical for CD4 receptor binding (Keane, J. et al. J Immunol. 160, 5945-5954 (1998)). The biologic activity of IL-16 resides in the N-terminus (Nicoll, J. et al. J Immunol. 163, 1827-1832 (1999)). To ensure exosomal membrane placement of this CD4+receptor targeting moiety, the C-terminus of IL-16 was fused with the extracellular domain of exosomal protein Lamp2b (Alvarez-Erviti, L. et al. Nat Biotechnol. 29, 341-345 (2011)). A stable cell line producing CD4+ receptor targeting exosomes harboring Tat (EXO^{CD4}-Tat) was generated. Compared to EXO-Tat, EXO^{CD4}-Tat led to a 20-fold increase in Tat protein delivery to rCD4+T lymphocytes (FIG. 6A and 6B).

Studies were carried out to determine whether this increased CD4 targeting ability led to greater reactivation of latent HIV-1 in primary cells. rCD+T cells were treated from another 3 ART-treated patients (Patient IDs: #230, #123 and #234 in Table 3) for 4 days and co-cultured the supernatants with MOLT-4 cells. EXO^{CD4}-Tat exosomes reactivated latent HIV-1 ex vivo in 3/3 individuals (FIG. 5D).

### Example 4

### EXO'-Tat Exosomes Showed No Significant Effect on T Cell Activation, Apoptosis or Cytokine Release

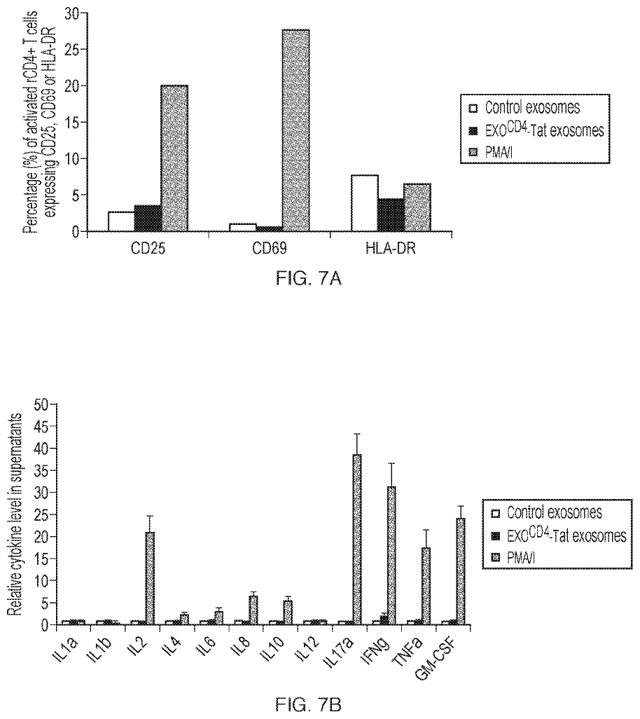

The potential toxicity of HIV-1 Tat is a concern when advancing the protein as a therapeutic. In cell model and murine animal systems, Tat expression is associated with bystander cell death, apoptosis and neuronal toxicity. The effect of EXO^{CD4}-Tat treatments (96 hrs) was quantified on immune activation and apoptotic parameters of primary rCD4+T lymphocytes in culture. Neither control nor EXO^{CD4}-Tat exosomes altered the activation status of rCD4+T lymphocytes as measured by FACS quantification of surface markers such as HLA-DR, CD-25 and CD-69 (FIG. 7A). Additionally, exosomal (control and EXO^{CD4}-Tat) treatment of rCD4+T cells had no significant effect on Annexin V. The expression levels of 12 pro-inflammatory cytokines and chemokines were measured in the culture media of rCD4+T cells treated with control or EXO^{CD4}-Tat exosomes. As seen in FIG. 7B, exosomal treatment had no appreciable effect on the cytokine panel compared to treatments with PMA/I which significantly increased the levels of IL-2, IL-17a, INF-γ, TNF-αand GM-CSF.

Exosome Targeting of CD4+ Receptor Expressing Cells

HAART regimens suppress viral replication to levels below the detection limit of current assays and have significantly decreased the morbidity and mortality associated with HIV-1 infection (Simon, V. et al. Lancet. 368, 489-504 (2006). Despite this clinical success, a reservoir of replication competent HIV-1 persists even after prolonged treatment thereby preventing viral cure (Dahabieh, M. et al. Annu Rev Med. 66, 407-421 (2015)). Current approaches to eradicate HIV-1 include pharmacologic approaches to reactivate latent virus with drugs such as histone deacetylase inhibitors (HDACi) and disulfiram (Rasmussen, T A. et al. Lancet HIV.1, e13-21 (2014), and Xing, S. et al. J Virol. 85, 6060-6064 (2011)). While these agents reverse HIV-1 latency in vitro, clinical administration has not been associated with significant reductions in viral burden in vivo. The ability of the HIV-1 protein Tat to activate viral transcription has been long known but few attempts have been made to harness the protein as a latency reversal agent (LRA). Potential toxicity and the practicality of generating sufficient amounts of clinical grade protein product are major limitations. The methods and compositions described herein overcome these limitations, which lays the foundation for establishing exosomal preparations as a clinically useful class of LRA.

The HEK293 cell line was as a factory for manufacturing exosomal Tat. While Tat is released by HIV-1 infected cells (Ensoli B. et al. J Virol. 67, 277-287 (1993), and Chang, H C. et al. AIDS. 11, 1421-1431 (1997)), the lack of appreciable secretion by the HEK293 cell line allowed us to modify expression vectors to maximize exosomal Tat concentration. An initial experimental challenge was faced in that exosomal localization compromised transactivating ability largely by sequestering Tat in non-nuclear compartments as seen in the first generation of constructs. Although the addition of a cymc NLS increased transactivation activity as quantified by in vitro models of viral latency, the efficacy in primary rCD4+T lymphocytes was poor with viral reactivation leading to replication competent virion progeny in 3/6 cases. This prompted targeting exosomes to specific cellular populations (perhaps the biggest challenge in exosomal therapeutics).

The experiments revealed that HEK293 produced exosomes that discharged cargo into 13% of purified CD4+T lymphocytes following co-culture for 24 hours. Recall that current data suggest that latent HIV-1 burden is surprisingly low after multi-year effective antiretroviral therapy with perhaps 1 in 1 million rCD4+T lymphocytes harboring replication competent HIV-1 (Siliciano, J D. et al. Curr Opin HIV AIDS. 8, 318-325 (2013), Laird, G M. et al. J Clin Invest. 125, 1901-1912 (2015), Xing. S. et al. Drug Discov Today. 18, 541-551 (2013), and Bullen, C K. et al. Nat Med. 20, 425-429 (2014)). LRAs based on an exosomal delivery platform need precision targeting with the ability to deliver cargo specifically to rCD4+T lymphocytes. A ligand/receptor interaction between IL-16/CD4+ receptor was made by expressing the C-terminal motifs of IL-16 responsible for CD4+ receptor binding in conjunction with the exosomal membrane protein Lamp2b. Surprisingly, these molecular manipulations increased rCD4+T lymphocyte uptake of exosomes by 20-fold with attendant increase in latency reversal potency. Exosomal targeting of rCD4+T lymphocytes led to viral reactivation and production of replication competent HIV-1 in 3/3 individuals tested.

Prior to the invention, a potential challenge in forwarding Tat based therapeutics was toxicity. The experiments did not find that exosomal Tat impacted cellular activation, levels of apoptosis or inflammatory cytokine release. These experiments were conducted using in vitro experimentation on primary rCD4+T lymphocytes to determine whether the latency reversal that was observed was a by-product of generalized immune stimulation or specific HIV-LTR activation. The results suggest the latter as a mechanism of action.

HIV-1 Tat is critical for the efficient replication of virus soon after chromosomal integration. The data described herein indicates that exosomal HIV-1 Tat is a safe and useful composition of purging the latent reservoir of infected cells.

### Example 5

### Exo-Tat Combined With a Dual-Affinity Re-Rargeting Antibody (DART) Molecule CD4-αCD3HA Led to the Elimination of HIV-1 Infected Primary CD4+T Cells

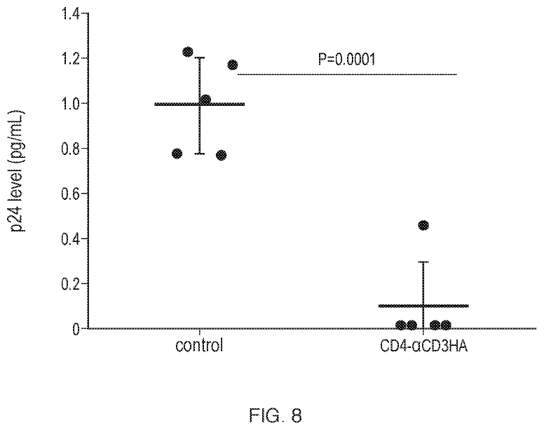

Primary CD4+T cells from HIV-1 infected patients contain latent HIV-1. Latent HIV-1 was activated from the CD4+T cells of 5 HIV-1 infected patients in the presence of CD4-αCD3HA with Exo-Tat exosomes. In the HIVE assays, CD4+T cells harboring reactivated HIV-1 were eliminated by autologous cytotoxic T cells indicating that combination of Exo-Tat and CD4-αCD3HA indeed reduce or eliminate HIV-1 reservoir (FIG. 8).

The following materials and methods were used in the studies described herein.

Cell Culture and Transfection

HEK293T cells were cultured in Dulbecco's modified Eagle's medium (Life Technologies) with 10% fetal bovine serum (FBS) (Thermo Scientific), 2 mM L-glutamine and non-essential amino acids (Life Technologies). U1 cells, primary human peripheral blood mononuclear cells (PBMCs) and CD4+T cells were cultured in RPMI Medium 1640 (Life Technologies) with 10% FBS. TZM-bl cells were cultured in Dulbecco's modified Eagle's medium with 10% PBS, 4 mM L-glutamine and non-essential amino acids. Adherent cells were trypsinized and re-seeded in culture plates 1 day before transfection or chemical treatment. HEK293T cells were transfected with Lipofectamine when cell confluency was ˜70%. TZM-bl cells were transfected with Lipofectamine 2000, GenJet Plus DNA Transfection Reagent (SignaGen Laboratories) or Lipofectamine LTX Plus Reagent (Life Technologies). For generation of exosomes and testing their function, regular FBS was replaced by exosome-depleted PBS (System Biosciences) in the culture media of HEK293T, U1 and TZM-bl cells, respectively.

Molecular Cloning

Using XPack Exosome Protein Engineering Technology (System Biosciences), the cDNA fragment encoding HIV-1 Tat protein with a myc nuclear localization signal fused to its C-terminus was subcloned into XPack CMV-XP-MCS-EF1-Puro Cloning Lentivector between enzyme sites BamHI and EcoRI. The original HIV-1 Tat plasmid was ordered from Addgene (Plasmid #14654) (Cujec, TP. et al. Genes Dev. 11, 2645-2657 (1997)). The generated construct was named EXO-Tat. The cDNA fragment encoding the C-terminal domain of interleukin 16 fused to the N-terminus of lysosome-associated membrane protein 2 variant b (lamp2b) was cloned into pCDH-EF1-MCS-T2A-Puro (System Biosciences) Cloning Lentivector between enzyme sites Swal and NotI. The generated construct was named pIL161amp2b. For the sequences of primers used for molecular cloning, please see Table 2 below. All constructs were sequenced at Yale Keck Biotechnology Resource Laboratory.

TABLE 2
Primers used for molecular cloning and qRT-PCR

| Primer name | Sequence |
|---|---|
| BamHI-Tat- | 5′GTTGGATCCAAATGGAGCCAGTAGATCCTAATC3′ |
| f | (SEQ ID NO: 21) |
| Tat-EcoRI- | 5′CGGAATTCTCAATCGAACGGATCTGTCTCTGTC3′ |
| r | (SEQ ID NO: 22) |
| First-s | 5′AGCTACAACAAGGCAAGGCTTGACCG3′ |
| | (SEQ ID NO: 23) |
| IL-16-r | 5′CTTGGACTGGAGGCTTTTTCTCCTGATGACAATC |
| | GTTCCACTGCCCATAGTCGAGTTAC3′ |
| | (SEQ ID NO: 24) |
| IL-16-f | 5′AGCCTCCAGTCCAAGGAAACCACAGCTGCTGGAG |
| | ACTCCGGCAGTGGATCTGGATCCG3′ |
| | (SEQ ID NO: 25) |
| SwaI-MES-f | 5′CTGATTTAAATGCCACCATGGTGTGCTTCCGCCT |
| | CTTCCCGGTTCCG3′ |
| | (SEQ ID NO: 26) |
| Lamp2b- | 5′TCTGCCCTCAGCGGCCGCGGAAGCGTAATCTGGC |
| NotI-r | ACATCGTATGGGTATC3′ |
| | (SEQ ID NO: 27) |

Generation of Lentivirus

A lentiviral packaging plasmid pPACKH1 (System Biosciences) was co-transfected into HEK293T cells with an empty vector (XPack CMV-XP-MCS-EF1-Puro Cloning Lentivector), pEXO-Tat, pIL161amp2b or pIL161am2b plus pEXO-Tat respectively at the ratio 2:1 to generate control lentiviruses, EXO-Tat lentiviruses, IL161amp2b lentiviruses or EXO^{CD4}-Tat lentiviruses. The supernatants of the transfected cells were collected at 48 h and 96 h post-transfection. The combined supernatants were filtered through a 0.45 μm Millex-HV Filter Unit (Merck Millipore). Lentiviruses were concentrated with PEG-itTM Virus Precipitation Solution (System Biosciences). The titers of viruses were determined with the UltraRapid Lentiviral Titer Kit (System Biosciences) following the manufacturer's instructions.

Generation of Stable Cell Lines

Four lines of cells stably expressing an empty vector (XPack CMV-XP-MCS-EF1-Puro Cloning Lentivector), pEXO-Tat, IL161amp2b or pEXO-Tat plus pIL161amp2b, respectively, were generated by transducing HEK293T cells with the above mentioned lentiviruses at MOI of 10. Three days post-transfection, puromycin 1 μg/ml was added to the culture medium to eliminate untransfected cells. To select cells with high Tat gene incorporation, puromycin concentration was increased to 5 μg/ml. The supernatants of stable cells were collected for isolation of exosomes. Tat protein expression was confirmed by western blot.

Exosome Isolation and Characterization

Stable cells were cultured in media with exosome depleted FBS. Supernatants of the stable cells were collected and used for isolation of exosomes using differential ultracentrifugation method: 300×g for 10 minutes, 2000×g for 30 minutes, 10,000×g for 30 minutes and then 100,000×g for 60 minutes; the last pellets were exosomes (Wen, S. et al. Leukemia. 30, 2221-2231 (2016)). Exosomes were washed once with plain RPMI medium. The exosomes were suspended in plain RPMI medium and stored either at 4° C. for 1-7 days or at −80° C. for further use. The number and size distribution of exosomes were determined on a NanoSight NS500 (Malvern Instruments, Malvern, UK) with a Syringe Pump.

Exosome Labeling and Uptake

Exosomes were directly labeled with 1 μM Vybrant Cell Tracers DiO (Life Technologies) by incubation for 30 minutes at 37° C. and then washed twice by ultracentrifugation at 100,000g for 1 hour in 1× phosphate-buffered saline (PBS) (Wen, S. et al. Leukemia. 30, 2221-2231 (2016)). Labeled exosomes were co-cultured with CD4+T cells for 24 hr and washed with PBS twice. Cells were analyzed on a BD Bioscience LSRII with FACS Diva 8.0.1 DIO fluorescence was excited from a 488 nm laser and detected through a 505 LP and 530/30 nm filter. Cells were separated from debris by utilizing a forward vs. side scatter dot plot. Twenty thousand events were collected for each sample. Analysis and figure preparation was performed using FlowJo V10 software.

Primary Antibodies and Primers

HA (Human influenza hemagglutinin), GFP (green fluorescent protein) and Alix mouse monoclonal antibodies were purchased from Cell Signaling Technology. GAPDH (0411) mouse monoclonal antibody and GAPDH (FL-335) rabbit polyclonal antibody were purchased from Santa Cruz Biotechnology. Lamp2b rabbit polyclonal antibody was from Abcam. Alexa Fluor 594 HA-tag mAb, human CD4 Alexa Fluor 488 mAb and human CD8 Alexa Fluor 647 mAb were from Thermo Fisher Scientific. All primers were ordered from Integrated DNA Technologies and are listed in Table 2.

Subcellular Fractionation

Subcellular fractionation was performed using Subcellular Protein Fractionation Kit for Cultured Cells (Thermo Scientific) according to the manufacturer's instructions. Briefly, cells were harvested and washed once with cold PBS. Cells were then suspended in CEB buffer and rotated at 4° C. for 10 min. After centrifugation at 500×g at 4° C. for 5 min, supernatant was collected as the cytoplasmic fraction. The pellets were suspended in MEB buffer and rotated at 4° C. for 10 min. After centrifugation at 3000×g at 4° C. for 5 min, supernatant was collected as the membranous fraction. The pellets were washed in MEM buffer twice and finally lysed in Pierce IP lysis buffer (Thermo Scientific) as the nuclear fraction.

Western Blot

Protein samples were prepared in Pierce IP lysis buffer (Thermo Scientific). Ten to 20 μg protein was mixed with NuPAGE LDS Samples Buffer (Life Technologies) and separated by 4-12% NuPAGE® Novex® 4-12% Bis-Tris gel electrophoresis and electroblotted to nitrocellulose membrane (Bio-Rad). Blotted membranes were probed with their respective primary antibodies, rotating at 4° C. overnight. Membranes were washed three times in TBS-T buffer and probed with secondary antibody (680 goat anti-rabbit IgG or IRDye800-conjugated Affinity Purified Anti-Mouse IgG, respectively) at room temperature for 1 h. Membranes were then washed three times in TBST buffer and direct infrared fluorescence detection was performed with a Licor Odyssey® Infrared Imaging System (Tang, X. et al. Cell. 131, 93-105 (2007)).

Luciferase Assay

Empty vector (EV), pTat, pXO-Tat or pEXO-Tat was transfected into TZM-bl cells when the cells were at about 60-70% confluence. Forty-eight hours post-transfection, luciferase activity was performed using the Dual-Glo® Luciferase Assay System (Promega). For each experiment, a control employing an empty vector was used and corrected luciferase values were averaged, arbitrarily set to a value of ‘1’ and served as a reference for comparison of fold-differences in experimental values (Tang, X. et al. Nucleic Acids Res. 38, 6610-6619 (2010)).

HIV-1 p24 Elisa Assay

Exosomes were added to U1 culture medium containing exosome-depleted FBS. Forty-eight hours after addition of exosomes, U1 cell culture media were collected and used for p24 Elisa assay using a p24 ELISA Kit (PerkinElmer) according the manufacturer's instructions. The analytical sensitivity of the kit is 17.1 pg/mL.

Study Subjects

HIV-1-infected individuals were enrolled in the study at The Miriam Hospital based on the criteria of suppressive antiretroviral therapy (ART) and undetectable plasma HIV-1 RNA levels (<50 copies per ml) for a minimum of 12 months. Characteristics of study participants are presented in Table 3. The study was approved by Lifespan Institutional Review Board. All research participants enrolled in the study provided written, informed consent prior to inclusion in this study.

TABLE 3
Characteristics of study participants

| Patient ID | Gender | Age | Race | CD4 | PVL | Year of Dx | <75 since | Medication |
|---|---|---|---|---|---|---|---|---|
| #211 | F | 51 | White | 884 | L20 | 1990 | July 2012 | Stribild |
| #219 | F | 45 | White | 782 | L20 | 2009 | June 2010 | Atriplia |
| #232 | F | 58 | White | 945 | L20 | 1990 | March 2009 | Atriplia |
| #111 | M | 56 | White | 650 | L20 | 2001 | April 2002 | Atriplia |
| #207 | F | 55 | Black | 1392 | L20 | 2001 | April 2003 | Ziagen, |
| | | | | | | | | Reyataz |
| | | | | | | | | Norvir |
| #112 | M | 63 | Black | 853 | L20 | 1998 | February 2001 | Truvada |
| | | | | | | | | Raltegravir |
| #204 | F | 44 | Black | 1503 | L20 | 1995 | August 2012 | Stribild |
| #225 | F | 65 | White | 892 | L20 | 1991 | March 2005 | Ziagen, |
| | | | | | | | | Sustiva, 3TC |
| #108 | M | 55 | Black | 435 | L20 | 1990 | December 2008 | Triumeq |
| #223 | F | 44 | White | 564 | L20 | 2005 | February 2007 | Norvir, |
| | | | | | | | | Truvada |
| | | | | | | | | Reyataz |
| #109 | M | 51 | White | 506 | L20 | 1986 | July 2012 | Reyataz |
| #230 | F | 49 | White | 803 | L20 | 1988 | August 2007 | Epzicom |
| | | | | | | | | Reyataz |
| #123 | M | 54 | White | 866 | L20 | 2005 | December 2012 | Norvir, |
| | | | | | | | | Epzicom, |
| | | | | | | | | Prezista |
| #234 | F | 31 | White | 865 | L20 | 2002 | March 2011 | Stribild |

Notes:
F = female,
M = male,
PVL = plasma viral load

Isolation and Culture of Resting CD4+T Cells

PBMCs from whole blood or buffy coats of healthy donors were purified using density centrifugation on a Ficoll-Hypaque (GE Healthcare) gradient. Resting CD4+T cells (CD4+, CD25-, CD69-, and HLA-DR-) were isolated by negative depletion using sequential combination of a human CD4+T cell isolation kit, a human CD25 MicroBeads II, a human CD69 MicroBead Kit II and a human anti-HLA-DR MicroBeads kit (Miltenyi Biotec) (Laird, GM. et al. J Clin Invest. 125, 1901-1912 (2015), and Bullen, CK. et al. Nat Med. 20, 425-429 (2014)). Cells were cultured in RPMI medium with 10% FBS at a concentration of 2×10⁶ cells per 0.6 mL for all experiments. For treatment, 50 μL of exosomes (1.8×10⁹ exosomes or 46.8 μg total protein) or 50 ng/ml PMA plus 1 μM ionomycin was added to 450 μL culture medium of rCD4 cells.

Measurement of Intracellular and Extracellular HIV-1 mRNA

Two million rCD4+T cells were treated with control exosomes (Exo-C), Tat exosomes (Exo-Tat) or PMA/I respectively for 4 days. The cells and supernatants were separated by centrifugation. Total RNA from the cells was used to detect intracellular HIV-1 mRNA, total RNA from supernatants was used to detect extracellular HIV-1 mRNA following the method established by Silicano lab (Laird, G M. et al. J Clin Invest. 125, 1901-1912 (2015)).

Flow Cytometry

rCD4+T cells were treated with control exosomes, engineered exosomes or PMA/I respectively for 48 h. The cells were subsequently used for measurement of T cell activation markers (CD25, CD69 and HLA-DR) or apoptosis marker Annexin V. For detecting T cell activation, FITC mouse anti-human CD25 (BD Pharmingen), APC mouse anti-human CD69 (BD Pharmingen), and PerCP-Cy 5.5 mouse anti-human HLA-DR (BD Pharmingen) were used respectively to stain the cells. For early apoptosis detection, PE Annexin V (BD Pharmingen) was used to stain the cells. Cells were analyzed on a BD Bioscience LSRII with FACS Diva 8.0.1. Analysis and figure preparation was performed using FlowJo V10 software.

HIV-1 p24 Antigen Assay

The Simoa p24 antigen assay is a 2-step digital immunoassay to measure the quantity of p24 using the Simoa HD-1 Analyzer and Single Molecule Array (Simoa) technology with an analytical sensitivity of 0.0074 pg/mL. Resting CD4+T cells were isolated from the PBMCs of blood of HIV-1 patients who were treated with ART for a period of time. Resting CD4+T cells were treated with exosomes or PMA/I for 4 days. The supernatants were collected and co-cultured with MOLT-4 cells to amplify HIV-1 virus. The supernatants from treated resting CD4+T cells or from co-cultured MOLT-4 cells were used to measure p24 concentration.

Immunocytochemistry

For viewing the random interaction between exosomes and cells, CD4+T cells were isolated from the PBMCs of a healthy donor using a Dynabeads® UntouchedTM Human CD4 T cells isolation kit (Invitrogen) and cultured in RPMI medium with 10% exosome depleted FBS. The cells were treated with control (EV) or Exo-Tat exosomes for 24 h. The cells were separated from culture medium by centrifugation and washed with PBS. The cells were fixed in 4% paraformaldehyde for 10 minutes and washed 3 times in PBS. Subsequently, cells were spread on Polysine® Microscope Slides (Thermo Scientific) and blocked in normal mouse serum (Thermo Fisher) for 1 hour. Cells were stained with HA mAb overnight and washed 3 three times in PBS. Cells were incubated with Alexa Fluor® 594 goat anti-mouse IgG (Life Technologies) for 1 hour and washed 3 more times before taking immunofluorescent images. For testing exosomes specifically targeting CD4+cells, PBMCs from healthy donors were treated with control exosomes, EXO-Tat exosomes or EXO'-Tat exosomes respectively for 24 h. The immunocytochemical procedure was similar but the fluorescence-labeled primary antibodies were utilized.

Immunofluorescent Imaging

Confocal images were acquired with a Nikon Clsi confocal (Nikon Inc. Mellville N.Y.) using diode lasers 402, 488, 561 and 638. Serial optical sections were performed with EZ-C1 computer software (Nikon Inc. Mellville, N.Y.). Each wavelength was acquired separately by invoking frame lambda. Z series sections were collected at 0.15 μm with a 100× Plan Apo lens and scan zoom of 2. Deconvolution and projections were performed in Elements version 3.2 (Nikon Inc. Mellville, N.Y.) computer software.

Cytokine Release Assay

Resting CD4+T cells were isolated from PBMCs of healthy donors and incubated with control exosomes, Exo-Tat/IL161amp2b exosomes or PMA/I respectively for 4 days. Culture supernatants were collected by centrifugation and used for cytokine assay. The concentration of 12 pro-inflammatory cytokines and chemokines (IL1 a, IL1β, IL2, IL4, IL6, IL8, IL10, IL12, IL17α, IFN-γ, TNFα and GM-CSF) in the supernatants were measured using a Multi-Analyte ELISArray Kit (Qiagen) following the manufacturer's instructions.

Statistical Analysis

Quantitative data were analyzed by unpaired Student's t test to compare two groups. Data are expressed as mean±standard error of mean. A p value<0.05 indicates statistical significance.

Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids, including ribonucleic acids (RNA) and deoxyribonucleic acids (DNA), and polynucleotides are a polymers of any length, including longer lengths, e.g., 200, 300, 500, 1000, 2000, 3000, 5000, 7000, 10000, 20000, 30000, 40000 etc. A nucleic acid will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506.

The term “bp” means base pairs.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire nucleic acid or polypeptide sequence or individual domains of a nucleic acid or polypeptide), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. In embodiments, the identify exists over a region that is about or at least about 20, 50, 100, 1000, 2500, 5000, 7500, 10000, 15000, 20000, 25000, or 30000 amino acids or nucleotides in length to about, less than about, or at least about 31000, 32000, 33000, 34000 or 35000 amino acids or nucleotides in length. Optionally, the identity exists over a region that is at least about 10 to about 100, about 20 to about 75, about 30 to about 50 amino acids or nucleotides in length. Optionally, the identity exists over a region that is at least about 50 amino acids in length, or more preferably over a region that is 100 to 500 or 1000 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

An example of algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. As will be appreciated by one of skill in the art, the software for performing BLAST analyses is publicly available through the website of the National Center for Biotechnology Information. In embodiments, BLAST and BLAST 2.0 are used, with the parameters described herein, to determine percent sequence identity for the nucleic acids and proteins. In embodiments, a BLAST algorithm involves first identifying high scoring sequence pairs (HSPs) by identifying short words of length W in the query sequence, which either match or satisfy some positive-valued threshold score T when aligned with a word of the same length in a database sequence. In embodiments, T is referred to as the neighborhood word score threshold (Altschul et al., supra). In embodiments, these initial neighborhood word hits act as seeds for initiating searches to find longer HSPs containing them. In embodiments, the word hits are extended in both directions along each sequence for as far as the cumulative alignment score can be increased. In embodiments, cumulative scores are calculated using, for nucleotide sequences, the parameters M (reward score for a pair of matching residues; always >0) and N (penalty score for mismatching residues; always <0). In embodiments, for amino acid sequences, a scoring matrix is used to calculate the cumulative score. In embodiments, extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. In embodiments, the BLAST algorithm parameters W, T, and X determine the sensitivity and speed of the alignment. For example, the BLASTN program (for nucleotide sequences) with defaults of a word length (W) of 11, an expectation (E) of 10, M=5, N=−4 and a comparison of both strands is used. For amino acid sequences, the BLASTP program with defaults of a word length of 3, and expectation (E) of 10 is used. In another example, the BLOSUM62 scoring matrix alignments (B) of 50, expectation (E) of 10, M=5, N=−4, and a comparison of both strands are used (see Henikoff & Henikoff, Proc. Natl. Acad. Sci. USA 89:10915 (1989)).

The terms “polypeptide,” “peptide” and “protein” are used interchangeably herein to refer to a polymer of amino acid residues. The term encompasses a string of amino acids conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a hybrid polymer of amino acid residues.

The term “amino acid” refers to a naturally occurring and synthetic amino acid, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For example, the codons GCA, GCC, GCG and GCU all encode the amino acid alanine. Thus, at every position where an alanine is specified by a codon, the codon can be altered to any of the corresponding codons described without altering the encoded amino acid. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical amino acid.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

Exemplary conservative substitutions are shown below:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

The term “disease” refers to any deviation from the normal health of an individual and includes a state when disease symptoms are present, as well as conditions in which a deviation (e.g., metabolic dysfunction or metabolic disorder) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. For example, the subject is a mammal such as a human subject. Other non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the term “about” in the context of a numerical value or range means ±10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also included in the range. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to a method for obtaining a reduction, alleviation or amelioration of pathological symptoms of a pathological condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of a disease, condition, or symptom of the disease or condition.

A method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. References to decreasing, reducing, or inhibiting include a change of 10%, 20%, 30%, 40%, 50%, 60%, 70%, 80%, 90% or greater as compared to a control level and such terms can include but do not necessarily include complete elimination of a disease or disorder. For example, severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” refer to the amount of an agent that is sufficient to achieve a desired effect, as described herein. The term “effective” when referring to an amount of cells or a therapeutic compound may refer to the quantity of the cells or the compound that is sufficient to yield a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to the amount of one or more compounds that is sufficient to result in or promote the production of the desired cell population, especially compared to culture conditions that lack the one or more compounds.

1. A composition comprising an exosome, wherein the exosome comprises a surface-exposed interleukin-16 (IL-16) polypeptide.

2. The composition of claim 1, wherein the exosome comprises a lysosomal-associated membrane protein (lamp)/IL-16 fusion protein.

3. The composition of claim 2, wherein the lamp protein comprises Lamp2b.

4. The composition of claim 1, wherein the exosome further comprises a latency reversal agent (LRA).

5. The composition of claim 4, wherein the latency reversal agent comprises an HIV Tat polypeptide.

6. The composition of claim 1, wherein the IL-16 polypeptide comprises the amino acid sequence of RRKS (SEQ ID ON: 1).

7. The composition of claim 1, wherein the exosome comprises a nuclear localization signal.

8. The composition of claim 7, wherein the nuclear localization signal comprises myc.

9. The composition of claim 1, wherein the exosome has a diameter from about 10 nm to about 1000 nm.

10. The composition of claim 1, wherein the exosome has a diameter from about 30 nm to about 100 nm.

11. A method for promoting viral transcription in a cell, the method comprising contacting an HIV-infected CD4+T cell with the composition of claim 4.

12. A method for preparing an exosome comprising a surface-exposed interleukin-16 (IL-16) polypeptide, the method comprising:
culturing cells in a medium, wherein the cells release the exosomes by secretion into the medium,
collecting the supernatant of medium,
fractionating the supernatant comprising the exosomes, and
isolating the exosomes.

13. The method of claim 12, wherein the cells comprise eukaryotic cells.

14. The method of claim 13, wherein the protein of interest comprises a viral protein.

15. The method of claim 14, wherein the viral protein comprises HIV Tat.

16. A method of treating a patient comprising human immunodeficiency virus-1 (HIV), comprising administering to the patient a composition comprising an exosome, wherein the exosome comprises a surface-exposed interleukin-16 (IL-16) polypeptide.

17. The method of claim 16, wherein the patient is administered the composition or intravenously, orally, or by inhalation.

18. The method of claim 16, wherein the patient comprises a human.

19. The method of claim 16, wherein the effective amount is an amount effective to promote viral transcription.

20. A method of treating a patient comprising human immunodeficiency virus-1 (HIV), comprising administering to the patient a composition comprising an adeno-associated virus encoding a LRA, wherein said LRA comprises a nuclear/exosomal localization modified Tat protein (Exo-Tat).


SEQUENCE LISTING (104 sequences)

SEQ ID NO: 1 - protein, 4 aa, Artificial Sequence
  IL16 polypeptide
RRKS

SEQ ID NO: 2 - protein, 9 aa, Artificial Sequence
  c-myc
PAAKRVKLD

SEQ ID NO: 3 - protein, 20 aa, Artificial Sequence
  nucleoplasmin
AVKRPAATKKAGQAKKKKLD

SEQ ID NO: 4 - protein, 25 aa, Artificial Sequence
  EGL-13
MSRRRKANPTKLSENAKKLAKEVEN

SEQ ID NO: 5 - protein, 9 aa, Artificial Sequence
  TUS protein
KLKIKRPVK

SEQ ID NO: 6 - protein, 12 aa, Artificial Sequence
  HIV1 Tat
GRKKRRQRRRAP

SEQ ID NO: 7 - protein, 20 aa, Artificial Sequence
  IL16 polypeptide
TIVIRRKSLQSKETTAAGDS

SEQ ID NO: 8 - protein, 631 aa, Homo sapiens
MDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPRRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 9 - DNA, 2076 bp, Homo sapiens
ctgctgctaccacaggaagacacagcagggagaagccctagtgcctctgccggctgcccaggacctggtatcggcccacagaccaagtcctccacagagggcgagccagggtggagaagagccagcccagtgacccaaacatccccgataaaacacccactgcttaagaggcaggctcggatggactatagctttgataccacagccgaagacccttgggttaggatttctgactgcatcaaaaacttatttagccccatcatgagtgagaaccatggccacatgcctctacagcccaatgccagcctgaatgaagaagaagggacacagggccacccagatgggaccccaccaaagctggacaccgccaatggcactcccaaagtttacaagtcagcagacagcagcactgtgaagaaaggtcctcctgtggctcccaagccagcctggtttcgccaaagcttgaaaggtttgaggaatcgtgcttcagacccaagagggctccctgatcctgccttgtccacccagccagcacctgcttccagggagcacctaggatcacacatccgggcctcctcctcctcctcctccatcaggcagagaatcagctcctttgaaacctttggctcctctcaactgcctgacaaaggagcccagagactgagcctccagccctcctccggggaggcagcaaaacctcttgggaagcatgaggaaggacggttttctggactcttggggcgaggggctgcacccactcttgtgccccagcagcctgagcaagtactgtcctcggggtcccctgcagcctccgaggccagagacccaggcgtgtctgagtcccctcccccaaggcggcagcccaatcagaaaactctcccccctggcccggacccgctcctaaggctgctgtcaacacaggctgaggaatctcaaggcccagtgctcaagatgcctagccagcgagcacggagcttccccctgaccaggtcccagtcctgtgagacgaagctacttgacgaaaagaccagcaaactctattctatcagcagccaagtgtcatcggctgtcatgaaatccttgctgtgccttccatcttctatctcctgtgcccagactccctgcatccccaaggaaggggcatctccaacatcatcatccaacgaagactcagctgcaaatggttctgctgaaacatctgccttggacacagggttctcgctcaacctttcagagctgagagaatatacagagggtctcacggaagccaaggaagacgatgatggggaccacagttcccttcagtctggtcagtccgttatctccctgctgagctcagaagaattaaaaaaactcatcgaggaggtgaaggttctggatgaagcaacattaaagcaattagacggcatccatgtcaccatcttacacaaggaggaaggtgctggtcttgggttcagcttggcaggaggagcagatctagaaaacaaggtgattacggttcacagagtgtttccaaatgggctggcctcccaggaagggactattcagaagggcaatgaggttctttccatcaacggcaagtctctcaaggggaccacgcaccatgatgccttggcaatcctccgccaagctcgagagcccaggcaagctgtgattgtcacaaggaagctgactccagaggccatgcctgacctcaactcctccactgactctgcagcctcagcctctgcagccagtgatgtttctgtagaatctacagcagaggccacagtctgcacggtgacactggagaagatgtcggcagggctgggcttcagcctggaaggagggaagggctccctacacggagacaagcctctcaccattaacaggattttcaaaggagcagcctcagaacaaagtgagacagtccagcctggagatgaaatcttgcagctgggtggcactgccatgcagggcctcacacggtttgaagcctggaacatcatcaaggcactgcctgatggacctgtcacgattgtcatcaggagaaaaagcctccagtccaaggaaaccacagctgctggagactcctag

SEQ ID NO: 10 - DNA, 60 bp, Artificial Sequence
  IL16
acgattgtcatcaggagaaaaagcctccagtccaaggaaaccacagctgctggagactcc

SEQ ID NO: 11 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 12 - DNA, 6603 bp, Homo sapiens
aagaaagagccccgcccctagtcttatgactcgcactgaagcgccgattcctggcttttgcaaggctgtggtcggtggtcatcagtgctcttgacccaggtccagcgagccttttccctggtgttgcagctgttgttgtaccgccgccgtcgccgccgtcgccgcctgctctgcggggtcatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctcaagactgcagtgcagatgacgacaacttccttgtgcccatagcggtgggagctgccttggcaggagtacttattctagtgttgctggcttattttattggtctcaagcaccatcatgctggatatgagcaattttagaatctgcaacctgattgattatataaaaatacatgcaaataacaagattttcttacctctcagttgttgaaacactttgcttcttaaaattgatatgttgaaactttaattcttttatcaatcccagcattttgagatcagtctttattaataaaacctgttctctttaatcagcttaaaatccaaagtgtcatatttactggtcctggagacaaacttgttcaaaagaacatcaacgtgcaatgttttaaggtctatcttaagaagccctggccaaattttgatcctaaccttgaagtatgccttgaacttattaacatggccattataagaataaaatatgtagttgtgtcttaatggaattaataaatgtcatttcactactggtgttctgtttcaatgtataaggactatagtgatttaaactcatcaatgtgcctttgcataaagttcattaaataaatattgatgtggtataaatgcccatcagatatgcttaaacttggttttcagttgaatgaagtagagaatgtcctcaggaccatcagcattttaaaggttatgtgacttttgctgatttctctgagttcaagttaagcatgaagttagtacctcaagcctgtgatttttccctagggatgatacagacccaagaggctacaacagaacttaaactggcttcgtaattagagtttttaagataattgtttgtttttcagcaatatagactgaaaagatccaagcatatttagccacttgcttttttgtttcttgttttgttcttctttggatgcctgattagtattgaaagatagaaatattctatgaactaattaggacagattgtgttgtgtttctctacctcatcttgttgatctctggagcattaaaatctatttagtgttgtcatcagtgtggtacttatgaaatgtaagctaacagcaatctcagaagggaggcagtgaagcatagcaactaggctcttgtttcttcaagatggcccctgtggggcagtgcatagatgggggtgtaaagagaagctgttggcattaaaatgagctagataatcagcccttgttgaagcatattccatggtataagagtagcacagacatgaaacatagataaagaaggaaggcttaatagactagaagacttccacattgaagtattattaacccattgtatgtatataggggcatgatcagagtctctataacttcctgattaacaatacagtgtatcttgttacccagctgtcagtctttgagagctttcagtaaaatatagtaaattctttcagcataggctaatgtgtggttactgagatgagtgttgtgtactcagaaccgtagcaacatttttatgaatggtaaaagtacaagaggaggaaaagttaaaattagaagaaaagtacaagttattgcttaatcataaatcacaccagataacacattttgttaatttcattagctattactggaaaggaccttaacgattatttacagaaaggggagtgaaattcattgaggttccatatcaagtgggcaacaaaactattactagcattttgataaaaattgcccctaatgaaatctagtcactcaacagtaaaacaacagctggtttacacttgaaattatgagatcagaattgggcactttgggcttccgtactatgttttgcttaagttttttttttaatactaatatgggctttttcagtagtaatataccaaaacacttctattttaatctctgtttgctacttcaaaacctaatcctcctcagatgggatcatgagcataagaggaaaagagaagagaatgaatacttgttgacctcttgatgtgtatcagatgctttagaaatgtaattgtatttaatcctcaaataccttataggtattattatccccttcttacagatgaggaaagtgaggcccagtttaaataacttgcccaaggtcctttggctagtactggaaggagtcaagatttaaacacagttctgtctgaatccagaactcaaaatctacattgcacatgttgctttcctggtggttcggtggaatggactgcaacgcattagatactgctgttattcttccaggccaccgctcagctaaaaataattgtgtgtgtgtgtatatatatatatatgtacacacacacacatatatatacacacacacatatatatacacatatacatatatatacacatatatatacacacacatatatatgtatatatatactacattcttgatcctaagtcttttttaacttaaattttattacttatacagaattcttatttatactttaattataggtgtgacgaagagaaagagagtagggaaatacacaggcagtggttttaagtgtagatgatggctccttaacccagtgtcattagataatcaaacctaaagtcttcccatattaggcaagcgcaattctctattttggacccttcccattcttcccttaccttctgcttttcgtactgaggaatttcgtgtgattttagataaagtgataatgagatattgagcaaataagaaaatagaggtaatgctataaaaaactaagctatgtacactttcaaaatgcatgtttcttgcatgctttttactacttaattgcattctttgctaatttcctttccttgctgtctgttcttttctaacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaatcagttaaatctagtgtttgtttgtttttttcaattagaagttacgtttccattggctaaaagccaggacatgctgtgcaatagattgtttaagatatgcagactaacttcagtgagttcctagctaacttgggcatgagtacacttatttaagacaaaatatattaggaccaatttttttctgttttttttcttcctttgttaaagtataattaaaagaaaaattgtggcttagaattttttaagtaaataatgattttaagcccctggatccaattatgaaagcatttttgctgatgtgtaattttatatgttacagttacttatattttactactttgatgttatttgcaaaatcaaaggtgttaaagaatttaacttgcttcaggaaataaattcaagaacatagtggattcattttcattggtggcagacacgaaatttggttcatgataagacttcctttccccacctcctgatcagcattatttaaatctgtatttttctgttagttaagaaagaaatggcttcatgatattgtatttaatagcaaaagtttggctgtcttcttcattactgttaatagctactatattttaacaaggagatttctttttttgttgttgttgttctagagtttggaatatactgattatctcagacttgacatttatactgaaggatgaagtaagacctccagctttttttaaaaaaggtgttgatttggaacacctgtatgggttatggtttattaaggttatggtttagaaagtttttttccctcagagccttaacttgttaagaaggttcatttatcctgcactgaaaacaaaaactctatatactttgtttgtgtgcctcctgcactctcccattccctatgtgaatatgctctagttgatatttttaatatattgatttcttttttctcacagcaacaagtgcttactctagaggttagtgggccctgatatgtcatcagtcagatgcctgcctagccaaagctggactaagattattctgtacatttgttgatcttgatatagacttatatccctgtagggactgctaatggctccggcttctggagtaaggtactggagaccactcatccctgtgtctgcttgattggttcagctgttgaattgcccttttatttggaagcagtgttgaagttgtctagggttcaaatggctgctttgtacacctgtcattagtataaggcagatgtttattttatcaagctattttatctctacatttaactaaaaacaaaagttcccaaagatctgccttcacttcagaaattttttttggattaaaaaaattaagcctgaaccttaaataaagtgagttggttattcattccaaggattaagtcccaatctacctctcagcacaatgcagaagctcaccactgtattgctgccattaactcatgccagaaccctttgccaataactggaattacaaatttttgttaaagaaaatttatcaagatctttctttactgccttctctatatgtacatctcaaaaacatgtacatctcaaaaactggagtagaaagttagattgctcaactacaactcctctagaactctatagctctgacatacagattcacactctcctctatttgctaagtatgtaaagaatgttttcttttaaaatgttctcttttgagaacaactgcttatttgttataaaagcatttggttaaaatgatgtcatcataaaaaacagtggctttgtttcaatacatatttttgagatgattatctagaagccagattaataaaatcagcttgtgaccttgctaagcatataaactggaaattcagatacattcaaaattatgggttcatttaaaagtgttctaccttttgggtatgagactaatatcactaattcctcaatagttatcatggctctatcttaattaattagaaaatatgtgtgtttaattctttgagaattaaaatagagaatattaacagagggttaaaaactgcttcaactccaataagataaaggaagctcaaaatctatgagctgagtgttcaattagctttgcctactgagttcaattttatgtcaatacaacagtggatcagacagtacgactttgaactggtgaatgtaaacaattgtttttcacctaagctgctttggaagaactgatgcttgctgctaactaaagttttggatgtatcgatttagagaaccaattaatacctgcaaaataaagcatactgtggtacttctgtttgatctagtatgtgtgattttagattgatggattaaaaattaataaagatcatacattccataccaaaaaaaaaaaaaaaa

SEQ ID NO: 13 - protein, 460 aa, Artificial Sequence
  IL-16/Lamp2b fusion protein
MVCFRLFPVPGSGLVLVCLVLGAVRSYAGNSTMGSGTIVIRRKSLQSKETTAAGDSGSGSGSGGSSLELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTLGSGYPYDVPDYA

SEQ ID NO: 14 - DNA, 1380 bp, Artificial Sequence
  nucleic acid sequence encoding IL-16/Lamp2b fusion protein
atggtgtgcttccgcctcttcccggttccgggctcagggctcgttctggtctgcctagtcctgggagctgtgcggtcttatgcaggtaactcgactatgggcagtggaacgattgtcatcaggagaaaaagcctccagtccaaggaaaccacagctgctggagactccggcagtggatctggatccggtggctcgagtttggaacttaatttgacagattcagaaaatgccacttgcctttatgcaaaatggcagatgaatttcacagttcgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgcctgctggctaccatggggctgcagctgaacatcactcaggataaggttgcttcagttattaacatcaaccccaatacaactcactccacaggcagctgccgttctcacactgctctacttagactcaatagcagcactattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgggaagcggatacccatacgatgtgccagattacgct

SEQ ID NO: 15 - protein, 111 aa, Artificial Sequence
  Tat
MEPVDPNLEPWKHPGSQPRTACNNCYCKKCCFHCYACFTRKGLGISYGRKKRRQRRRAPQDSQTHQASLSKQPASQSRGDPTGPTESKKKVERETETDPFDYPYDVPDYAA

SEQ ID NO: 16 - DNA, 333 bp, Artificial Sequence
  Tat cDNA
atggagccagtagatcctaatctagagccctggaagcatccaggaagtcagcctaggactgcttgtaacaattgctattgtaaaaagtgttgctttcattgctacgcgtgtttcacaagaaaaggcttaggcatctcctatggcaggaagaagcggagacagcgacgaagagctcctcaggacagtcagactcatcaagcttctctatcaaagcaacccgcctcccagtcccgaggggacccgacagggcccacggaatcgaagaagaaggtggagagagagacagagacagatccgttcgattacccatacgatgttccagattacgctgcc

SEQ ID NO: 17 - protein, 101 aa, Artificial Sequence
  Tat
MEPVDPNLEPWKHPGSQPRTACNNCYCKKCCFHCYACFTRKGLGISYGRKKRRQRRRAPQDSQTHQASLSKQPASQSRGDPTGPTESKKKVERETETDPFD

SEQ ID NO: 18 - DNA, 303 bp, Artificial Sequence
  Tat cDNA
atggagccagtagatcctaatctagagccctggaagcatccaggaagtcagcctaggactgcttgtaacaattgctattgtaaaaagtgttgctttcattgctacgcgtgtttcacaagaaaaggcttaggcatctcctatggcaggaagaagcggagacagcgacgaagagctcctcaggacagtcagactcatcaagcttctctatcaaagcaacccgcctcccagtcccgaggggacccgacagggcccacggaatcgaagaagaaggtggagagagagacagagacagatccgttcgat

SEQ ID NO: 19 - protein, 86 aa, Artificial Sequence
  Tat
MEPVDPNLEPWKHPGSQPRTACNNCYCKKCCFHCYACFTRKGLGISYGRKKRRQRRRAPQDSQTHQASLSKQPASQSRGDPTGPTE

SEQ ID NO: 20 - DNA, 258 bp, Artificial Sequence
  Tat cDNA
atggagccagtagatcctaatctagagccctggaagcatccaggaagtcagcctaggactgcttgtaacaattgctattgtaaaaagtgttgctttcattgctacgcgtgtttcacaagaaaaggcttaggcatctcctatggcaggaagaagcggagacagcgacgaagagctcctcaggacagtcagactcatcaagcttctctatcaaagcaacccgcctcccagtcccgaggggacccgacagggcccacggaa

SEQ ID NO: 21 - DNA, 33 bp, Artificial Sequence
  BamHI-Tat-f Primer
gttggatccaaatggagccagtagatcctaatc

SEQ ID NO: 22 - DNA, 33 bp, Artificial Sequence
  Tat-EcoRI-r  Primer
cggaattctcaatcgaacggatctgtctctgtc

SEQ ID NO: 23 - DNA, 26 bp, Artificial Sequence
  First-s Primer
agctacaacaaggcaaggcttgaccg

SEQ ID NO: 24 - DNA, 59 bp, Artificial Sequence
  IL-16-r Primer
cttggactggaggctttttctcctgatgacaatcgttccactgcccatagtcgagttac

SEQ ID NO: 25 - DNA, 58 bp, Artificial Sequence
  IL-16-f Primer
agcctccagtccaaggaaaccacagctgctggagactccggcagtggatctggatccg

SEQ ID NO: 26 - DNA, 47 bp, Artificial Sequence
  SwaI-MES-f Primer
ctgatttaaatgccaccatggtgtgcttccgcctcttcccggttccg

SEQ ID NO: 27 - DNA, 50 bp, Artificial Sequence
  Lamp2b-NotI-r Primer
tctgccctcagcggccgcggaagcgtaatctggcacatcgtatgggtatc

SEQ ID NO: 28 - protein, 9 aa, Artificial Sequence
  HIV1 Tat
RKKRRQRRR

SEQ ID NO: 29 - protein, 10 aa, Artificial Sequence
  Tat peptide
MGCINSKRKD

SEQ ID NO: 30 - protein, 7 aa, Artificial Sequence
  NLS motif PDX1
RRMKWKK

SEQ ID NO: 31 - protein, 7 aa, Artificial Sequence
  NLS motif QKI-5
RVHPYQR

SEQ ID NO: 32 - protein, 24 aa, Artificial Sequence
  NLS motif HCDA
KRPACTLKPECVQQLLVCSQEAKK

SEQ ID NO: 33 - protein, 7 aa, Artificial Sequence
  NLS motif SV40 LrgT
PKKKRKV

SEQ ID NO: 34 - protein, 7 aa, Artificial Sequence
  NLS motif H2B
GKKRSKA

SEQ ID NO: 35 - protein, 6 aa, Artificial Sequence
  NLS motif v-Rel
KAKRQR

SEQ ID NO: 36 - protein, 8 aa, Artificial Sequence
  NLS motif Amida
RGRRRRQR

SEQ ID NO: 37 - protein, 5 aa, Artificial Sequence
  NLS Motif Amida
RKRRR

SEQ ID NO: 38 - protein, 9 aa, Artificial Sequence
  NLS Motif RanBP3
PPVKRERTS

SEQ ID NO: 39 - protein, 10 aa, Artificial Sequence
  NLS Motif Pho4p
PYLNKRKGKP

SEQ ID NO: 40 - protein, 17 aa, Artificial Sequence
  NLS Motif DNAhelicaseQ1
CYGSKNTGAKKRKIDDA

SEQ ID NO: 41 - protein, 9 aa, Artificial Sequence
  NLS Motif LEF-1
KKKKRKREK

SEQ ID NO: 42 - protein, 9 aa, Artificial Sequence
  NLS Motif TCF1
KKKRRSREK

SEQ ID NO: 43 - protein, 32 aa, Artificial Sequence
  NLS Motif TCPTP
  misc_feature (10)..(27): x is any amino acid
RKRIREDRKXXXXXXXXXXXXXXXXXXRKRKR

SEQ ID NO: 44 - protein, 15 aa, Artificial Sequence
  NLS Motif BDV-P
  X (5)..(8): x is any amino acid
RRERXXXXRPRKIPR

SEQ ID NO: 45 - protein, 14 aa, Artificial Sequence
  NLS Motif Act/inhBA
KKKKKEEEGEGKKK

SEQ ID NO: 46 - protein, 8 aa, Artificial Sequence
  NLS Motif BDV-P
PRPRKIPR

SEQ ID NO: 47 - protein, 13 aa, Artificial Sequence
  NLS Motif BDV-P
PPRIYPQLPSAPT

SEQ ID NO: 48 - protein, 20 aa, Artificial Sequence
  NLS Motif TR2
KDCVINKHHRNRCQYCRLQR

SEQ ID NO: 49 - protein, 15 aa, Artificial Sequence
  NLS Motif THOV NP
  X (3)..(11): x is any amino acid
KRXXXXXXXXXKTKK

SEQ ID NO: 50 - protein, 11 aa, Artificial Sequence
  NLS Motif Polyoma VP1
APKRKSGVSKC

SEQ ID NO: 51 - protein, 12 aa, Artificial Sequence
  NLS Motif HIV-1 Rev
RQARRNRRRRWR

SEQ ID NO: 52 - protein, 18 aa, Artificial Sequence
  NLS Motif Rex
MPKTRRRPRRSQRKRPPT

SEQ ID NO: 53 - protein, 17 aa, Artificial Sequence
  NLS Motif SRY
KRPMNAFIVWSRDQRRK

SEQ ID NO: 54 - protein, 5 aa, Artificial Sequence
  NLS Motif SRY
PRRRK

SEQ ID NO: 55 - protein, 17 aa, Artificial Sequence
  NLS Motif SOX9
KRPMNAFMVWAQAARRK

SEQ ID NO: 56 - protein, 5 aa, Artificial Sequence
  NLS Motif SOX9
PRRRK

SEQ ID NO: 57 - protein, 30 aa, Artificial Sequence
  NLS Motif AS Vintegrase
  X (1)..(1): X is amino acid K, A or R
  X (30)..(30): X is amino acid K or A
XTPIQKHWRPTVLTEGPPVKIRIETGEWEX

SEQ ID NO: 58 - protein, 9 aa, Artificial Sequence
  NLS Motif NS5A
PPRKKRTVV

SEQ ID NO: 59 - protein, 13 aa, Artificial Sequence
  NLS Motif DNAse EBV
YKRPCKRSFIRFI

SEQ ID NO: 60 - protein, 13 aa, Artificial Sequence
  NLS Motif DNAse EBV
LKDVRKRKLGPGH

SEQ ID NO: 61 - protein, 5 aa, Artificial Sequence
  NLS Motif AdenoE la
KRPRP

SEQ ID NO: 62 - protein, 23 aa, Artificial Sequence
  NLS Motif YstDNAApola/HProTa
RKRKKKRAAEDDEDDDVDTKKQK

SEQ ID NO: 63 - protein, 8 aa, Artificial Sequence
  NLS Motif Tst1/Oct6
GRKRKKRT

SEQ ID NO: 64 - protein, 6 aa, Artificial Sequence
  NLS Motif 20S proteasome
KKKQKK

SEQ ID NO: 65 - protein, 13 aa, Artificial Sequence
  NLS Motif ProtHsc9
REKKEKEQKEKCA

SEQ ID NO: 66 - protein, 13 aa, Artificial Sequence
  NLS MOtif Prot Hsci
LEKKVKKKFDWCA

SEQ ID NO: 67 - protein, 13 aa, Artificial Sequence
  Prot Hsc3
  X (5)..(5): X is Q or G
TEKKXKSILYDCA

SEQ ID NO: 68 - protein, 13 aa, Artificial Sequence
  NLS Motif Ta a
SDKKVRSRLIECA

SEQ ID NO: 69 - protein, 7 aa, Artificial Sequence
  NLS Motif Pax-QNR
LKRKLQR

SEQ ID NO: 70 - protein, 7 aa, Artificial Sequence
  NLS Motif HuntDis prot
RRKGKEK

SEQ ID NO: 71 - protein, 13 aa, Artificial Sequence
  NLS Motif MyoD
CKRKTTNADRRKA

SEQ ID NO: 72 - protein, 11 aa, Artificial Sequence
  NLS Motif MyoD
VNEAFETLKRC

SEQ ID NO: 73 - protein, 30 aa, Artificial Sequence
  NLS Motif Opaque2
MPTEERVRKRKESNRESARRSRYRKAAHLK

SEQ ID NO: 74 - protein, 21 aa, Artificial Sequence
  NLS Motif CTP
KVNSRKRRKEVPGPNGATEED

SEQ ID NO: 75 - protein, 6 aa, Artificial Sequence
  NLS Motif HCV
PRRGPR

SEQ ID NO: 76 - protein, 14 aa, Artificial Sequence
  NLS Motif HCV
PRGRRQPIPKARQP

SEQ ID NO: 77 - protein, 17 aa, Artificial Sequence
  NLS Motif P110RB1
KRSAEGGNPPKPLKKLR

SEQ ID NO: 78 - protein, 20 aa, Artificial Sequence
  NLS Motif Hpoly(ADP)poly
  X (4)..(14): X is any amino acid
KRKXXXXXXXXXXXKKKSKK

SEQ ID NO: 79 - protein, 11 aa, Artificial Sequence
  NLS Motif VirD2-Nterm
EYLSRKGKLEL

SEQ ID NO: 80 - protein, 20 aa, Artificial Sequence
  NLS Motif VirD2-Cterm
PKRPRDRHDGELGGRKRARG

SEQ ID NO: 81 - protein, 16 aa, Artificial Sequence
  NLS Motif Nucloplasmin
KRPAATKKAGQAKKKK

SEQ ID NO: 82 - protein, 18 aa, Artificial Sequence
  NLS Motif Nucleolin
KRKKEMANKSAPEAKKKK

SEQ ID NO: 83 - protein, 19 aa, Artificial Sequence
  NLS Motif ICP-8
RKRAFHGDDPFGEGPPDKK

SEQ ID NO: 84 - protein, 21 aa, Artificial Sequence
  NLS Motif Nab2
  X (4)..(6): X is any amino acid
  X (11)..(16): X is any amino acid
GGGXXXKNRRXXXXXXRGGRN

SEQ ID NO: 85 - protein, 15 aa, Artificial Sequence
  NLS Motif M9
YNNQSSNFGPMKGGN

SEQ ID NO: 86 - protein, 9 aa, Artificial Sequence
  NLS Motif LscMyc
PAAKRVKLD

SEQ ID NO: 87 - protein, 17 aa, Artificial Sequence
  NLS Motif HumKprotien
KRPAEDMEEEQAFKRSR

SEQ ID NO: 88 - protein, 11 aa, Artificial Sequence
  NLS Motif FluA
  X (2)..(2): X is any amino acid
  X (9)..(10): X is any amino acid
SXGTKRSYXXM

SEQ ID NO: 89 - protein, 13 aa, Artificial Sequence
  NLS Motif Mata
MNKIPIKDLLNPG

SEQ ID NO: 90 - protein, 7 aa, Artificial Sequence
  NLS Motif Polyoma Lrg-T
PKKARED

SEQ ID NO: 91 - protein, 7 aa, Artificial Sequence
  NLS Motif Polyoma Lrg-T
VSRKRPR

SEQ ID NO: 92 - protein, 8 aa, Artificial Sequence
  NLS Motif S V40 VP 1
APTKRKGS

SEQ ID NO: 93 - protein, 7 aa, Artificial Sequence
  NLS Motif S V40 VP2
PNKKKRK

SEQ ID NO: 94 - protein, 12 aa, Artificial Sequence
  NLS Motif Polyoma VP2
EEDGPQKKKRRL

SEQ ID NO: 95 - protein, 8 aa, Artificial Sequence
  NLS Motif c-myb
PLLKKIKQ

SEQ ID NO: 96 - protein, 8 aa, Artificial Sequence
  NLS Motif N-myc
PPQKKIKS

SEQ ID NO: 97 - protein, 7 aa, Artificial Sequence
  NLS Motif P53
PQPKKKP

SEQ ID NO: 98 - protein, 9 aa, Artificial Sequence
  NLS Motif c-erb-A
SKRVAKRKL

SEQ ID NO: 99 - protein, 10 aa, Artificial Sequence
  NLS Motif Yeast SKI3
IKYFKKFPKD

SEQ ID NO: 100 - protein, 7 aa, Artificial Sequence
  NLS Motif L29
KTRKHRG

SEQ ID NO: 101 - protein, 7 aa, Artificial Sequence
  NLS Motif L29
KHRKHPG

SEQ ID NO: 102 - protein, 8 aa, Artificial Sequence
  NLS Motif Max
PQSRKKLR

SEQ ID NO: 103 - protein, 19 aa, Artificial Sequence
  NLS Motif L3
  X (10)..(15): X is any amino acid
HRKYEAPRHXXXXXXPRKR

SEQ ID NO: 104 - protein, 9 aa, Artificial Sequence
  NLS Motif Dyskerin
KKEKKKSKK